(19) United States
(12) Patent Application Publication
(10) Pub. No.: US 2026/0367078 A1
(43) Pub. Date: Nov. 19, 2026

(54) METHODS OF IDENTIFYING, IMAGING, AND CHARACTERIZING BACTERIAL SPORES USING LANTHANIDE-BETA-DIKETONES

(71) Applicant: TEMASEK LIFE SCIENCES LABORATORY LIMITED, Singapore (SG)

(72) Inventors: Shen-Yi Ian CHEONG, Singapore (SG); Ajitha SUNDARESAN, Singapore (SG)

(73) Assignee: TEMASEK LIFE SCIENCES LABORATORY LIMITED, Singapore (SG)

(21) Appl. No.: 19/142,487

(22) PCT Filed: Dec. 26, 2023
(86) PCT No.: PCT/SG2023/050860

§ 371 Date: Jun. 23, 2025

(30) Foreign Application Priority Data
Dec. 27, 2022 (SG) 10202260603W

(51) Int. Cl. G01N 33/569; G01N 1/30; G01N 21/17; G01N 33/58

(52) U.S. Cl. CPC G01N 33/56911; G01N 1/30; G01N 21/17; G01N 33/582; G01N 2001/302; G01N 2333/195; G01N 2333/32; G01N 2333/33

(57) ABSTRACT

The invention relates to methods, compositions, and kits for staining, imaging, identifying, and/or characterizing one or more bacterial spores in a sample by contacting the sample with a probe having a formula Ln[BD]n wherein Ln is a member of the lanthanide series of elements, BD is a beta-diketone, and n is a whole number integer, to stain one or more bacterial spores in the sample; and detecting a fluorescent signal from the one or more stained bacterial spores in the sample using a fluorescence microscope and visualizing the morphology of the one or more bacterial spores in the sample using a phase-contrast microscope.

### CROSS-REFERENCE TO RELATED APPLICATIONS

This application is a 35 U.S.C. § 371 National Stage of International Patent Application No. PCT/SG2023/050860, filed Dec. 26, 2023, claiming benefit from Singapore Patent Application No. 10202260603W, filed Dec. 27, 2022, the disclosures of which are incorporated herein in their entirety by reference, and priority is claimed to each of the foregoing.

### TECHNICAL FIELD

The present disclosure relates to the field of imaging and detection. In particular, a non-toxic lanthanide-beta diketonate probe and related methods for imaging and characterizing bacterial spores in situ are disclosed.

### BACKGROUND

Bacteria belonging to the genera Bacillus and Clostridium are ubiquitous in nature, and have the ability to initiate endospore formation upon sensing unfavorable environmental conditions. Such spores may persist for many years, with the longest known survival being plausibly 250 million years. These long-lived dormant spores remain viable and can germinate into vegetative cells when favorable conditions return. Bacterial spores may cause a host of medically significant infections, especially foodborne illnesses (e.g. Bacillus cereus, Clostridium perfringens, C. botulinum) and nosocomial infections (e.g. Clostridiodes difficile). It has been postulated that certain bacterial spores may, without the requirement for further engineering, be used as bioweapons, or even make their way into space thereby compromising life-detection experiments and inadvertently transform extra-terrestrial ecosystems.

Apart from their threat as infectious agents, spore detection is a concern for space exploration, since contamination of foreign planets with life that originated on Earth might compromise life-detection experiments and inadvertently transform the extra-terrestrial ecosystem. Similar spore contamination concerns also exist in the food, pharmaceutical manufacturing, and medical equipment industries, where sterility is paramount. In all of these fields of study, there is a long-felt and unmet need for quick detection and triage of suspected spore material.

Spore detection methods may be classified as direct or indirect. The earliest attempts at bacterial spore detection relied primarily on ultraviolet (UV) absorbance to reveal the presence of dipicolinic acid (DPA), a universal constituent of bacterial spores. In contrast, recent methods have shifted towards detecting DPA using fluorescence spectroscopy. The current state-of-the-art is based on detecting the fluorescent complex formed between spore-released DPA and the Terbium(III) cation (Tb³⁺). Visualizing this signal, which is diffusible and not localized to the spore, requires immobilizing the spore on a diffusion-retarding substrate such as agarose, and a fluorescent microscope able to accommodate the low excitation wavelength (~270 nm) of the DPA/Tb³⁺ complex. Due to these limitations, DPA detection is mostly practical as an indirect indicator of germinated spores at the population level.

The detection limit for such indirect detection is 10⁵ CFU/ml, although this can be improved to 10³ CFU/ml if dodecylamine and heating at 60° C. is used to maximize DPA release. Since only viable spores with intact DPA reservoirs contribute to the fluorescent signal, pre-germinated spores which have released their DPA will be undetectable. Hence, DPA/Tb³⁺ fluorescence does not reveal if a negative sample was devoid of spores or full of germinated spores. Especially in the second case, this might lead to underestimation of the pathogenic threat since germinated spores can easily transform into the infectious vegetative forms. Colorimetric reagents for DPA detection have also been developed, such as Erbium-Pyrocatechol Violet and Betanin, but these suffer similar constraints, in addition to having a poorer limit of detection (~10⁶ CFU/ml).

Bacterial endospores have a robust morphology which prevents many dyes from penetrating the spore structure. For this reason, conventional techniques have required permeabilizing the spore structure to facilitate staining. For instance, the Schaeffer-Fulton assay requires permeabilization through heat fixation and steaming to enable the entry of malachite green into the spore. Among the fluorescent stains, some will stain only either the dormant form (ThT) or the germinated form (SYTO-16). The lack of staining for either the dormant or germinated form makes visualization and enumeration of non-staining spores difficult or impossible. The remaining fluorescent agents stain both dormant and germinant forms equally and without visual distinction (DAPI, AO), rendering them unable to distinguish between dormant and germinated spores. Finally, an issue specific to malachite green and ThT is that their associated protocols are inherently toxic to the germinating spore, making it impossible to use these methods for the live imaging of germination events.

Thus, there is a need for a non-destructive method that allows for the direct detection and characterization of bacterial spores. There is also a need for a method of instantaneous detection of bacterial spores and their germination status. There is also a need for live imaging of bacterial spores and their germination status.

### SUMMARY OF THE INVENTION

The present disclosure provides compositions, methods, and kits for using lanthanide-beta-diketones to fluorescently label bacterial spores instantaneously in situ, without further processing. Given its low toxicity to spores, and its ability to differentiate between dormant and germinated forms, this method also allows for the live imaging of germinating single spores.

In one aspect, the present invention is a method for live imaging one or more bacterial spores in a sample comprising contacting the sample with a probe having a formula Ln[BD]ₙ wherein Ln is a member of the lanthanide series of elements, BD is a beta-diketone, and n is a whole number integer, to stain one or more bacterial spores in the sample; and detecting a fluorescent signal from the one or more stained bacterial spores in the sample using a fluorescence microscope.

Another aspect of the present invention is a method for distinguishing germination states of one or more bacterial spores in situ comprising contacting a sample comprising one or more bacterial spores with a probe having a formula Ln[BD]ₙ, wherein Ln is a member of the lanthanide series of elements, BD is a beta-diketone, and n is a whole number integer, to stain one or more bacterial spores in the sample; live imaging fluorescent signal of the sample using a fluorescence microscope; and distinguishing between dormant bacterial spores and germinating or germinated bacterial spores in the sample. In some embodiments the dormant bacterial spores have a fluorescence signal characterized by a donut-shaped fluorescence profile with a central hollow, and germinating or germinated bacterial spores have a central core filled with fluorescence wherein Ln is a member of the lanthanide series of elements, BD is a beta-diketone.

In other embodiments, the present invention is a method for real-time live imaging germination of bacterial spores comprising contacting one or more bacterial spores with a germinant probe solution comprising one or more germinants and a probe having a formula Ln[BD]ₙ, wherein Ln is a member of the lanthanide series of elements, BD is a beta-diketone, and n is a whole number integer, to stain one or more bacterial spores in the sample to form a mixture; mounting the mixture on a slide; visualizing the morphology of the one or more bacterial spores on the slide using time-lapse phase-contrast microscopy over time; and imaging fluorescent signals from the one or more bacterial spores on the slide using time-lapse fluoresce microscopy over time.

The method for live imaging one or more bacterial spores in a sample and the method for distinguishing germination states of one or more bacterial spores in situ may further comprise performing signal detection using a USB powered microscope, a digital microscope, a confocal microscope, a Raman microscope, or a bright-field microscope. Accordingly, the advantageous features of the inventive method include that (i) it is instantaneous, (ii) no special processing is involved, (iii) it does not destroy spores-permits live imaging of a germinating spore, (iv) stains spores, distinguishes between dormant and germinated spores, (v) low-cost USB-powered microscope can also be used (although details of spores cannot be visualised), the low cost visualization can be used to provide at least a preliminary on-the site-result of a sample.

In some aspects, the real-time live imaging of the germination of the one or more bacterial spores comprises three phase-contrast microscopy phases comprising: 1) a phase bright stage and a 2) phase gray stage of spore germination correlated with the most intense fluorescent signal being localized to regions exterior to the core of the spore, and 3) a phase dark stage of spore germination correlated with the most intense fluorescent signal being localized to the core region of the spore, wherein the phase dark stage indicates bacterial spore germination.

In some embodiments, any one of the above methods may be used in food pathogen detection, environmental monitoring, quality assurance, or microbiology research for monitoring spore bioburden in food or pharmaceutical samples, for monitoring spore bioburden in the environment, or for monitoring spore bioburden as a quality assurance step in an industrial process. In some aspects, the industrial process is in producing self-healing concrete.

In some embodiments, the sample comprises a plurality of bacterial spores, and the method further comprises quantifying the plurality of bacterial spores in the sample. In some embodiments, the imaging and/or monitoring comprises producing a video of the sample to observe changes in germination over time.

The present invention also includes a kit comprising a germination probe having a formula Ln[BD]ₙ wherein Ln is a member of the lanthanide series of elements, BD is a beta-diketone, and n is a whole number integer in a first container, and a germinant in a second container. In some aspects, the germinant (or compound that supports germination) is L-cysteine, hypoxanthine, oxyrase, sodium glycocholate, taurocholate, glycine, L-alanine, L-valine, D-valine, or D-cycloserine. In some aspects, the kit comprises a plurality of germinants, wherein each germinant is provided in a separate container.

The present invention also includes a composition comprising a germination probe having a formula Ln[BD]ₙ wherein Ln is a member of the lanthanide series of elements, BD is a beta-diketone, and n is a whole number integer and a germinant, wherein the germinant (or compound that supports germination) is L-cysteine, hypoxanthine, oxyrase, sodium glycocholate, taurocholate, glycine, L-alanine, L-valine, D-valine, or D-cycloserine. In some aspects, L-valine or D-valine may be used as a germinant for C. novyi-NT species.

Other features and characteristics of the subject matter of this disclosure, as well as the methods of operation, functions of related elements of structure and the combination of parts, and economies of manufacture, will become more apparent upon consideration of the following description and the appended claims, all of which form a part of this specification.

In one aspect, the present invention is an apparatus comprising means for live imaging one or more bacterial spores in a sample in which the one or more bacterial spores have been contacted with a probe having a formula Ln[BD]ₙ. In some aspects, Ln may be a member of the lanthanide series of elements, BD is a beta-diketone, and n is a whole number integer, to stain one or more bacterial spores in the sample. In some aspects, the means for live imaging is a camera, microscope, image capture device, video capture device, or combination thereof. A camera, microscope, image capture device, video capture device, or combination thereof is understood by persons of ordinary skill in the art as having a structure that performs imaging.

In some aspects, an apparatus may comprise a means for distinguishing germination states of one or more bacterial spores in a sample in situ. In some aspects, the sample may comprise one or more bacterial spores stained with a probe having a formula Ln[BD]ₙ. In some aspects, Ln is a member of the lanthanide series of elements, BD is a beta-diketone, and n is a whole number integer. In some aspects, the apparatus may be configured to obtain a live image fluorescent signal of the sample using fluorescence microscopy and distinguish between dormant bacterial spores and germinating or germinated bacterial spores in the sample.

In some aspects, the dormant bacterial spores have a fluorescence signal that may be characterized by a donut-shaped fluorescence profile with a central hollow. In certain aspects, the germinating or germinated bacterial spores have a central core filled with fluorescence.

In some aspects, an apparatus may comprise a means for real-time live imaging germination of bacterial spores that have been contacted with a germinant probe solution comprising one or more germinants and a probe having a formula Ln[BD]ₙ.

In some aspects, Ln is a member of the lanthanide series of elements, BD is a beta-diketone, and n is a whole number integer, to stain one or more bacterial spores in the sample to form a mixture. In some aspects, the apparatus may comprise a slide configured to hold said mixture.

In some aspects, the apparatus may comprise a time-lapse phase-contrast microscope configured to visualize the bacterial spores in the mixture and image fluorescent signals from the bacterial spores on the slide. In some aspects, the real-time live imaging of the germination of the one or more bacterial spores comprises three phase-contrast microscopy phases comprising: 1) a phase bright stage and a 2) phase gray stage of spore germination correlated with the most intense fluorescent signal being localized to regions exterior to the core of the spore, and 3) a phase dark stage of spore germination correlated with the most intense fluorescent signal being localized to the core region of the spore, wherein the phase dark stage indicates bacterial spore germination.

### BRIEF DESCRIPTION OF DRAWINGS

FIG. 1A shows fluorescence images of germinated C. novyi-NT spores stained with different proportions of Eu and TTA. FIG. 1B shows fluorescence images of germinated C. novyi-NT spores stained with different concentrations of Eu[TTA]₃. FIG. 1C shows germinated C. novyi-NT spores stained with 1.4 mM Eu[TTA]₃(left), 1.4 mM EuCl₃ (center) and 4.2 mM TTA (right). The top row represents wide-field phase contrast images and the bottom row represents fluorescence images.

FIG. 2A shows dormant (left) and germinated (right) spores of Clostridium novyi-NT, Clostridium septicum, Bacillus subtilis, Clostridioides difficile and Bacillus thuringiensis stained with 1.4 mM Eu[TTA]₃. The top represents wide-field phase contrast images and the bottom represents fluorescence images. FIG. 2B shows DPA (1017 cm⁻¹) and Europium (1531 cm⁻¹) Raman peak mapping across the longest spore axis for dormant and germinated C. novyi-NT spores. The average Eu Raman peak intensity profiles (Top) of spores stained with Eu[TTA]₃ (left) and EuCl₃ (right) and average DPA Raman peak intensity profiles (Bottom) of spores stained with Eu[TTA]₃ (left) and EuCl₃ (right) respectively (N=6 for each sample).

FIG. 3A shows pseudo colored confocal images of Eu[TTA]₃ (1.4 mM) stained dormant (left) and germinated (right) spores—C.novyi-NT (Row 1), C. septicum (Row 2), C. difficile (Row 3), B. subtilis (Row 4) and B. thuringiensis (Row 5). FIGS. 3B-3D show pseudo-colored confocal images of co-staining of Eu[TTA]₃ (Green) with membrane dye Nile Red (Red), co-staining of Eu[TTA]₃ (Green) with DNA staining dye DAPI (Blue), and co-staining of Eu[TTA]₃ (Green) with FITC-dextran 3 k-5 k (Red), respectively.

FIG. 4 shows dormant and germinated C. novyi-NT spores treated with 1.4 mM of the respective lanthanide-TTA complex. Images represent widefield phase contrast (Rows 1 and 3) and fluorescence (Rows 2 and 4) images of dormant (Top) and germinated (Bottom) C. novyi NT spores stained with 1.4 mM Eu[TTA]₃ (Column 1) Sm[TTA]₃ (Column 2), Dy[TTA]₃ (Column 3) and Tb[TTA]₃ (Column 4).

FIG. 5 shows dormant and germinated C. novyi-NT spores stained with 1.4 mM of different Eu beta-diketone complexes. Images represent widefield phase contrast (Rows 1 and 3) and fluorescence (Rows 2 and 4) images of dormant (Top) and germinated (Bottom) C. novyi -NT spores stained with 1.4 mM Eu[BTFA]₃ (Column 1) Eu[NTFA]₃ (Column 2), Eu[AA]₃ (Column 3), Eu[TriFAA]₃ (Column 4) and Eu[TetraFAA]₃ (Column 5).

FIG. 6A shows pre-germinated C. novyi-NT and B. subtilis spores treated with Eu[TTA]₃ (1.4mM) for 15 min and plated on BHI-FBS or LB agar plates respectively. Data represents the average of two independent experiments and error bars represent standard deviations. FIGS. 6B and 6C show time-lapse of Eu[TTA]₃-stained C. novyi-NT spores during germination with L-cysteine, hypoxanthine and oxyrase and Eu[TTA]₃-stained B. subtilis P 1 A1 spores during germination with L-alanine, respectively. Each time panel consists of the corresponding phase contrast (top) and fluorescence image (bottom). Insets below show magnified fluorescence images of a germinating spore along with the 3D surface plot projection of a single germinating spore's fluorescence using the Interactive 3D Surface plot plugin in ImageJ.

FIG. 7A shows representative phase contrast and fluorescence images along with their Raman spectra for a single phase bright, phase gray and phase dark spore. Average Raman spectra of spores at the three different stages of germination (N=20 for each stage). Average Raman intensity peak profiles of DPA (1017 cm⁻¹) and Eu (1531 cm⁻¹) peaks for phase bright, phase gray and phase dark spores (N=20 for each stage). FIG. 7B shows Eu[TTA]₃ (1.4 mM) added to pre-germinated wild-type P1A1 and cwlD mutant spores. Left represents wild-type spores while right represents cwlD mutant spores. Top panel represents phase contrast images while Bottom panel represents fluorescence images. FIG. 7C shows dormant C. novyi-NT spores treated with de-coating buffer followed by treatment with cortex lysis buffer containing lysozyme and stained with Eu[TTA]₃ (1.4 mM). Column 1 depicts Eu[TTA]₃ stained untreated dormant spores, Column 2 represents Eu[TTA]₃ stained de-coated spores, Column 3 represents lysozyme treated dormant spores. Column 4 represents lysozyme treated de-coated spores. Top panel depicts phase contrast images while the bottom panel depicts fluorescence images.

FIG. 8 (Top) RGB images of dormant and germinated Clostridium novyi-NT and B. subtilis spores stained with malachite green for 15 min and counter-stained with safranin. (Bottom) Phase contrast (Rows 1,3) and fluorescence (Rows 2,4) images of dormant (left) and germinated (right) spores of Clostridium novyi-NT and Bacillus subtilis stained with DAPI (5 μM) and acridine orange (9.3 mg/ml) for 30 min respectively.

FIG. 9. Fluorescence images represent dormant (left) and germinated (right) spores of Clostridium novyi-NT, Clostridium septicum, Clostridioides difficile, Bacillus subtilis, and Bacillus thuringiensis stained with 1.4 mM Eu[TTA]_{3.}

FIG. 10 a 2.5% (w/v) milk sample spiked with dormant C. novyi-NT spores and aliquots of this sample were stained with Eu[TTA]₃ (1.4 mM). Images represent phase contrast (Top) and fluorescence (Bottom) images of C. novyi-NT spores in milk matrix stained with (left) and without (right) Eu[TTA]_{3.}

FIG. 11 shows 3D maximum intensity projection images of Z-stack confocal images of dormant (left) and germinated (right) C. novyi-NT (Top) and C. septicum (Bottom) spores from FIG. 3.

FIG. 12 shows a table comparison of performance of various spore staining agents.

FIG. 13A shows Z-average intensity projection images of Z-stack confocal images of C. novyi-NT (left) and B. subtilis (right) germinating cultures in BHI-FBS oxyrase media and LB media respectively, both stained with Eu[TTA]₃ (green). FIG. 13B shows single Z-plane confocal images of early sporulating overnight culture of B. cereus in 2×SG sporulation media stained with Eu[TTA]₃ (green). Scale bar 2 μm. FIG. 13C shows Phase contrast (Top) and fluorescence (Bottom) images of B. cereus spores in 2.5% (v/v) milk matrix stained with (left) and without (right) Eu[TTA]₃. Scale bar: 2 μm.

### DETAILED DESCRIPTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or embodiments so described.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” or “consisting of” would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The present disclosure includes detecting any bacterial spores and is not limited by the exemplary bacterial spore types that were used in the experiments. The experiments serve as proof of concept to support use of the inventive methods for detection of bacterial spores generally. Non-limiting examples of such bacterial spores include, for example, B. subtilis, a well characterized model for bacterial spore biology; B. thuringiensis, an insect pathogen which expresses pore-forming toxins with insecticidal applications and also a close relative of B. anthracis and sometimes serves as a surrogate in research studies; B. cereus, a common food borne pathogen capable of releasing toxins that can cause food poisoning and related illness; C. novyi-NT, a gas gangrene bacteria derived from Clostridium novyi type A by removing its indigenous bacteriophage, making it genetically equivalent to Clostridium botulinum type C; C. difficile, a nosocomial infectious agent transmitted in hospital settings; and C. septicum, a pathogen of livestock and is also known to be associated with infections in patients which harbor existing (and often occult) hematological and gastrointestinal malignancies. These proof of concept bacterial spore types were selected and confirmed that the present invention is effective across a diverse cross-section of endospore-forming bacteria.

The germination process for bacterial spores is rudimentarily divided into three sequential stages, termed phase bright, phase gray and phase dark. These terms describe the luminance of the spore observed under phase contrast microscopy. Phase darkening and the release of the spore biomarker DPA are both hallmark features of bacterial spore germination.

Additionally, the bacterial spore contains sieve-like layers of varying permeability that selectively limit the access of small and large molecules. Among these sieves, the cortex layer is known to be the final physical barrier to the bacterial spore inner membrane. In the normal course of germination, this cortical barrier is hydrolyzed by enzymes.

The present disclosure provides compositions, methods, and kits for using lanthanide-beta-diketonate to fluorescently label bacterial spores instantaneously in situ, without further processing. Given its low toxicity to spores, and its ability to differentiate between dormant and germinated forms, this method also allows for the live imaging of germinating single spores. As such, in some aspects, the present invention provides a method that does not require an incubation period.

In one aspect, the present invention is a method for live imaging one or more bacterial spores in a sample comprising contacting the sample with a probe having a formula Ln[BD]ₙ wherein Ln is a member of the lanthanide series of elements, BD is a beta-diketone, and n is a whole number integer, to stain one or more bacterial spores in the sample; and detecting a fluorescent signal from the one or more stained bacterial spores in the sample using a fluorescence microscope.

Another aspect of the present invention is a method for distinguishing germination states of one or more bacterial spores in situ comprising contacting a sample comprising one or more bacterial spores with a probe having a formula Ln[BD]ₙ, wherein Ln is a member of the lanthanide series of elements, BD is a beta-diketone, and n is a whole number integer, to stain one or more bacterial spores in the sample; live imaging fluorescent signal of the sample using a fluorescence microscope; and distinguishing between dormant bacterial spores and germinating or germinated bacterial spores in the sample. In some embodiments the dormant bacterial spores have a fluorescence signal characterized by a donut-shaped fluorescence profile with a central hollow, and germinating or germinated bacterial spores have a central core filled with fluorescence wherein Ln is a member of the lanthanide series of elements, BD is a beta-diketone.

In other embodiments, the present invention is a method for real-time live imaging germination of bacterial spores comprising contacting one or more bacterial spores with a germinant probe solution comprising one or more germinants and a probe having a formula Ln[BD]ₙ, wherein Ln is a member of the lanthanide series of elements, BD is a beta-diketone, and n is a whole number integer, to stain one or more bacterial spores in the sample to form a mixture; mounting the mixture on a slide; visualizing the morphology of the one or more bacterial spores on the slide using time-lapse phase-contrast microscopy over time; and imaging fluorescent signals from one or more bacterial spores on the slide using time-lapse fluoresce microscopy over time.

In some embodiments, Raman microscopy may distinguish dormant spores showing a double peak profile from germinated spores showing a single peak profile. In some aspects, the method further comprises detecting peaks in the double peak profile comprising an inner membrane region and a trough between the peaks coinciding with core region of the spore, and comprising detecting the single peak profile comprising the core of the spore. In some aspects, there is no statistically significant shift in the Raman profile of the Ln levels of the Ln[BD]ₙ probe throughout germination. In some embodiments, Dipicolinic Acid (DPA) is not present when the single peak corresponding to the Ln[BD]ₙ probe in the core region of the spore is present.

In some embodiments, the signal detection is performed using confocal microscopy, wherein the optical plane slice images through dormant spores show a hollow region devoid of any fluorescent signal corresponding to the core region of the spore, and wherein the optical plane slice images through germinated spores show a fluorescent signal in the center of the spore corresponding to the core region of the spore.

The method for live imaging one or more bacterial spores in a sample and the method for distinguishing germination states of one or more bacterial spores in situ to may further comprise performing signal detection using a USB powered microscope, a digital microscope, a confocal microscope, a Raman microscope, or a bright-field microscope.

In some aspects, the real-time live imaging of the germination of the one or more bacterial spores comprises three phase-contrast microscopy phases comprising: 1) a phase bright stage and a 2) phase gray stage of spore germination correlated with the most intense fluorescent signal being localized to regions exterior to the core of the spore, and 3) a phase dark stage of spore germination correlated with the most intense fluorescent signal being localized to the core region of the spore, wherein the phase dark stage indicates bacterial spore germination.

In some embodiments, the germinants (or compound that supports germination) used are one or more of L-cysteine, hypoxanthine, oxyrase, sodium glycocholate, taurocholate, glycine, L-alanine, L-valine, D-valine, or D-cycloserine. In some embodiments, the Ln in the Ln[BD]ₙ probe is Europium, Samarium, Dysprosium, or Terbium. In some embodiments, the BD in the Ln[BD]ₙ probe is at least one selected from the group consisting of thenoyltrifluoroacetone (TTA), 1-(-2-Naphthoy1)-3,3,3-trifluoroacetone (NTFA), 4,4,4-Trifluoro-1-phenyl-1,3-butanedione (BTFA), Acetylacetone (AA), 1,1,1 Trifluoroacetylacetone (TriFAA), and 1,1,5,5 Tetrafluoroacetylacetone (TetraFAA). In some embodiments, the n in the Ln[BD]ₙ probe is 1 to 5, or 2 to 4, or 3.

In some embodiments, the probe is at least one selected from the group consisting of Eu[TTA]₃, Sm[TTA]₃, Dy[TTA]₃, Tb[TTA]₃, Eu[BTFA]₃, Eu[NTFA]₃, Eu[AA]₃, Eu[TriFAA]₃, and Eu[TetraFAA]₃. In some embodiments, the probe is Europium (III) thenoyltrifluoroacetone (Eu[TTA]₃).

In some aspects, the ratio of Ln to BD in the Ln[BD]ₙ probe is in the range of 9:1 to 1:9. In some aspects, the concentration of the probe is in the range of 0.7 mM to 10 mM, or 1 mM to 5 mM, or 1.4 mM to 2.8 mM, the ranges supporting selection of any concentration or ranges of concentration ranges from within the ranges.

In some embodiments, the probe binds to spores from at least one of the Bacillus genera, the Clostridium genera, or the Clostridioides genera. In some aspects, the probe binds spores from at least one of B. subtilis, B. thuringiensis, B. cereus, C. novyi-NT type A, C. difficile, or C. septicum.

In some embodiments, the treatment with the probe does not significantly affect spore viability, outgrowth, or colony-forming ability when compared to untreated spores. In some aspects, wherein a change in colony forming units (CFU) of the bacterial spores before and after treatment with the probe is less than 0.5 log CFU.

In some aspects, staining with the probe occurs instantaneously or near instantaneously. In some aspects, the bacterial spores are visualized immediately after the contacting step.

In some aspects, any one of the above methods does not include a step of spore permeabilization or a step of heating. In some aspects, a fluorescent brightener is not used. In some aspects, the methods of the present disclosure do not include using malachite green.

In some aspects, spore cortex hydrolysis precedes probe entry into the core.

In some embodiments, the fluorescence microscope is equipped with a 4′,6-diamidino-2-phenylindole (DAPI) long-pass filter.

In some aspects, the further comprising adding one or more surfactants to the sample. In some aspects, one or more surfactants may include Triton X-100 (2-[4-(2,4,4-trimethylpentan-2-yl)phenoxy]ethanol), a polysorbate, e.g., polysorbate 20 (TWEEN® 20), sodium dodecyl sulfate, or a combination thereof. In some aspects, any one of the above methods may further comprise contacting the sample with one or more additional probes or dyes and detecting one or more additional signals. In some aspects, one or more of the above methods further comprises co-staining bacterial spores with Nile Red, DAPI, fluorescein isothiocyanate (FITC)-dextran, or a combination thereof.

In some embodiments, any one of the above methods may be used in food pathogen detection, environmental monitoring, quality assurance, or microbiology research. for monitoring spore bioburden in food or pharmaceutical samples, for monitoring spore bioburden in the environment, or for monitoring spore bioburden as a quality assurance step in an industrial process. In some aspects, the industrial process is in producing self-healing concrete.

In some embodiments, the sample comprises a plurality of bacterial spores, and the method further comprises quantifying the plurality of bacterial spores in the sample. In some embodiments, the imaging and/or monitoring comprises producing a video of the sample to observe changes in germination over time.

The present invention also includes a kit comprising a germination probe having a formula Ln[BD]ₙ wherein Ln is a member of the lanthanide series of elements, BD is a beta-diketone, and n is a whole number integer in a first container, and a germinant in a second container. In some aspects, the germinant is L-cysteine, hypoxanthine, oxyrase, sodium glycocholate, taurocholate, glycine, L-alanine, L-valine, D-valine, or D-cycloserine. In some aspects, the kit comprises a plurality of germinants, wherein each germinant is provided in a separate container.

The present invention also includes a composition comprising a germination probe having a formula Ln[BD]ₙ wherein Ln is a member of the lanthanide series of elements, BD is a beta-diketone, and n is a whole number integer and a germinant, wherein the germinant is L-cysteine, hypoxanthine, oxyrase, sodium glycocholate, taurocholate, glycine, L-alanine, L-valine, D-valine, or D-cycloserine.

The invention of the present disclosure will be more readily understood by reference to the following examples, which are included merely for purposes of illustration of certain aspects and embodiments of the present invention, and are not intended to limit the scope of disclosed invention.

### EXAMPLES

The following examples are intended to exemplify the present disclosures and are not limitations of the claimed invention. All molecules, compositions, methods, assays, and results disclosed in the examples and other sections of the specification, figures, and claims form part of the disclosure of the invention.

### Example 1

Clostridium novyi-NT sporulation. Spores of Clostridium novyi-NT strains were generated according to the method reported in Dang et al. 2001. Briefly, the organisms were grown anaerobically in Gaspak™ anaerobic jar at 37° C., pH 7.4 in a medium containing 5 g Na₂HPO₄, 30 g polypeptone peptone, 0.5 g L-cysteine, 10 g maltose, 50 g dried cooked meat particles (Difco) per 1 liter and 10% v/v FBS. After 3 weeks in this medium, spores settled in the cooked meat particle layer. Spores were further purified from contaminating vegetative forms on a discontinuous Percoll gradient. The quality of spores was confirmed by phase contrast microscopy and contained >99% phase bright spores. The spore concentration was counted using a Neubauer counting chamber (Petroff Marenfield) with special depth and was adjusted approximately to the order of 5×10⁹ CFU/ml.

Bacillus sporulation. For B. subtilis, B. cereus and B. thuringiensis spores, sporulation was done based on the protocol from (Nicholson and Setlow, 1990). Bacillus strains were inoculated on a Luria Bertani (LB) agar plate overnight at 37° C. The next day, single colonies were inoculated into 2×SG medium (30 ml, pH 7) and allowed to grow on a MaxQ 8000 Orbital shaker (200 rpm, 37° C.) till it reached an OD₆₀₀ value of 0.3-0.5. This culture (25 ml) was then re-inoculated in a 21 Erlenmeyer flask containing 2×SG media (225 ml, pH 7) and covered with a gas permeable membrane (Breathe Easyr®) for allowing sufficient aeration. The flask was then incubated in a Gerhardt orbital shaker (130 rpm, 37° C.) for 44 hours for all the mutant strains and 92 hours for wild-type strains. The harvested spores were washed in 1×PBS followed by isopynic centrifugation using self-forming Percoll gradient (70% for mutant strains and 90% for wild-type strains) in a Beckmann-Coulter Ultra centrifuge (JS 13.1 rotor, 15000 rcf, 30 min, 4° C.). The majority of the phase-bright spores were obtained as the bottom fraction for the wild-type strain and as the top fraction for the mutant strains except for B. cereus where they were obtained in the top fraction. These fractions were repeatedly washed with 1× Phosphate buffer saline (PBS) and stored in 4° C. until further use. The quality of spores was confirmed by phase contrast microscopy and contained >99% phase bright spores. The spore concentration for all strains was counted using a Neubauer counting chamber (Petroff Marenfield) with special depth and was adjusted approximately to the order of 10⁹ CFU/ml for each strain except for B. cereus spores, which was adjusted to the order of 10¹⁰ CFU/ml.

Clostridium septicum sporulation. C. septicum spores were purified according to the protocol by Dang et al, 2001, with some modifications. All steps were performed inside a Plas labs anaerobic chamber. Briefly, overnight cultures of C. septicum were diluted 50X into 100 ml of BHI-S media containing 0.05% L-Cysteine and incubated until the OD 600 was 1.5-3 before the entire culture was added into 900ml of sporulation media (0.05% L-cysteine, 3% bacto polypeptone, 5% dehydrated cooked meat medium, and 10% fetal bovine serum) and incubated at 37° C. for five days. Spores were purified from vegetative cells on an 80% discontinuous Percoll gradient at 15000 rcf for 30 min in a Beckman Avanti J-20 XP high performance centrifuge. The obtained spores were washed twice, resuspended in water and stored at 4° C. until further use. Spore quality was checked by phase contrast microscopy and was found to contain >99% phase bright spores. The spore concentration for all strains was counted using a Neubauer counting chamber (Petroff Marenfield) with special depth and was adjusted approximately to the order of 10⁹ CFU/ml for each strain.

Clostridoides difficile sporulation. C. difficile sporulation was performed based on the protocol from Edwards et al, 2016. Briefly, stock concentrations of C. difficile bacteria were streaked on a 0.1% Taurocholate-BHI-S agar plate (BHI 37 gm/l, Yeast extract 5 gm/l, Agar 15gm/l, L-Cysteine 1 gm/l) and incubated overnight at 37° C. inside the anaerobic chamber. The next morning, one colony from the overnight plate was picked and inoculated in 5 ml of 0.1% Taurocholate-BHI-S liquid media. After 6 hours, 250 ul of culture was plated on ten 0.1% Taurocholate-BHI-S agar plates. After 6 days, the spores were harvested by washing the plates with 1×PBS. The cells were then pelleted by centrifugation (3900 rcf, 15 min) and re-suspended in 1×PBS (5 ml). The spores were then harvested by Percoll (90%) isopycnic centrifugation at 15000 rcf, 30 min, 4° C.. The bottom fraction was collected and re-suspended in 2 ml 1×PBS and stored at 4° C. until further use. The quality of spores was confirmed by phase contrast microscopy and contained >99% phase bright spores. The spore concentration for all strains was counted using a Neubauer counting chamber (Petroff Marenfield) with special depth and was adjusted approximately to the order of 10⁹ CFU/ml for each strain.

### Example 2

### Preparation of Europium Thenoyl Trifluoroacetone and Other Lanthanide Complexes.

Lanthanide TTA complex stock solutions were prepared by mixing equal volumes (200 μl) of stock solutions of respective Lanthanide chloride (3 mM) and TTA (9 mM) in 3% ethanol. The mixed solutions were vortexed at maximum speed for 30 min to aid complexation. The Eu:TTA ratio was used at 1:3. The claimed concentration range was found to be within the optimum concentration range which may allow for an adequate signal while minimizing the amount of organic solvent needed for TTA solubilization for live cell imaging. For other Europium beta-diketone complexes, all were prepared in 3% ethanol except NTFA which was prepared in 50% ethanol.

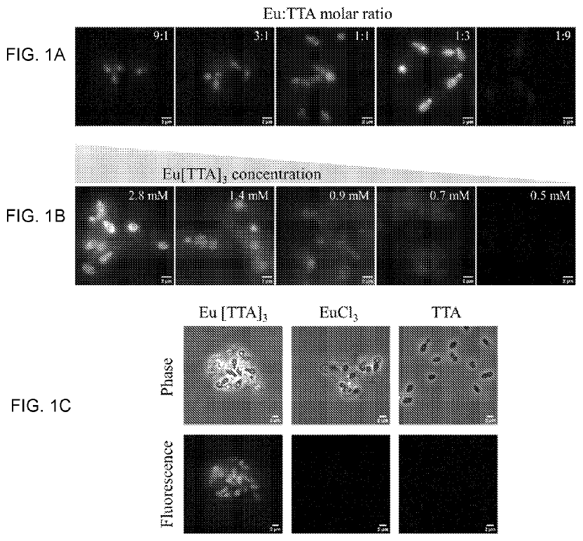

For the Eu: TTA stoichiometry determination, stock solutions of EuCl₃ (300 mM) and TTA (9 mM) in 3% ethanol were prepared. The EuCl₃ was then diluted in 3% ethanol such that the Eu: TTA molar ratios of 9:1, 3:1, 1:1, 1:3 and 1:9 were obtained. Equal volumes (200 ul) of these Eu solutions and TTA (9 mM) were mixed and vortexed for 30 min to form the EuTTA complexes with different stoichiometric ratios. As illustrated in FIG. 1A imaged using fluorescence microcopy, Eu: TTA complexes with ratios ranging from 9:1 to 1:9 with fixed TTA molar concentrations were used to stain germinated C. novyi-NT spores.

For the optimum concentration determination of Eu[TTA]₃ respective stock solutions of EuCl₃ (6 mM, 3 mM, 2 mM, 1.5 mM and 1 mM) and TTA (18 mM, 9 mM, 6 mM, 4.5 mM and 3 mM) were prepared in 3% ethanol. Equal volumes (200 μl) of these respective Eu and TTA solutions were mixed and vortexed for 30 min to form the EuTTA complexes with different concentrations at a stoichiometry of 1:3 Eu:TTA. The concentration was limited by the solubility of TTA in the solvent (3% ethanol in water). As illustrated in FIG. 1B, imaged using fluorescence microcopy, germinated C. novyi-NT spores, were stained with Eu[TTA]₃ with a concentration of 2.8 mM, 1.4 mM, 0.9 mM, 0.7 mM, 0.5 mM.

The exposure time was set as 10 milliseconds (ms) for fluorescence mode and 100 ms for phase contrast mode. The display histogram range was adjusted for images from each experiment to ensure better visibility. Images were obtained using the Zeiss Observer 7 microscope with 1600× (FIG. 1A, FIGS. 1B) and 1000× (FIG. 1C) magnification with a long pass DAPI filter. The scale bar is 2 μm.

Spores stained separately with either Eu or TTA did not fluoresce, demonstrating both Eu and TTA are necessary for spore fluorescence. FIG. 1C shows germinated C. novyi-NT spores stained with 1.4 mM Eu[TTA]₃(left), 1.4 mM EuCl₃ (center) and 4.2 mM TTA (right). The top row represents wide-field phase contrast images while the bottom row represents fluorescence images.

### Example 3

Eu[TTA]₃ assay for dormant and germinated spores. C. novyi-NT spores were germinated using a cocktail mixture of L-cysteine (100 mM), hypoxanthine (0.1 mM) and Oxyrase (1:50). C. septicum spores were germinated in sodium glycocholate (9.3 mM) and oxyrase (1:50). C. difficile spores were germinated in sodium taurocholate (8.5 mM), glycine (45 mM) and oxyrase (1: 50). B. subtilis spores were germinated in L-alanine (20 mM) after 1 hour of heat activation at 70° C. while B. thuringiensis was first treated with D-cycloserine (9.7 mM) at 70° C., 30 min to inactivate conversion of the germinant L-alanine to the non-germinant D-alanine and then germinated with L-alanine (93.5 mM).

All end-point germination assays were conducted at 37° C. for 30 min (except for cwlD spores which were germinated for 15 hours) after which the spores were centrifuged at 3900 rcf for 5 min and re-suspended in 1×PBS (for Bacillus spores) or 1×PBS+Oxyrase (for Clostridium spores). To 3.5 μμl of spores, 50 μl of an Ln-BD complex was added and an aliquot of this sample (10 μl) was used for instant imaging.

Microscopy was done on an inverted wide-field microscope (Zeiss Axiovert 200M with a Photometrics Coolsnap HQ2 camera, 1000× magnification for all bacteria except C. difficile and Zeiss Observer 7 with a Hamamatsu ORCA flash 4.0 V2 camera, 1600× magnification for C. difficile). The imaging was done on a 100× Plan Apochromat Ph3 objective (N.A. 1.4, Oil objective lens) and a DAPI long pass filter (Excitation filter-365/12 BP; Dichroic 395; Emission filter 397 LP).

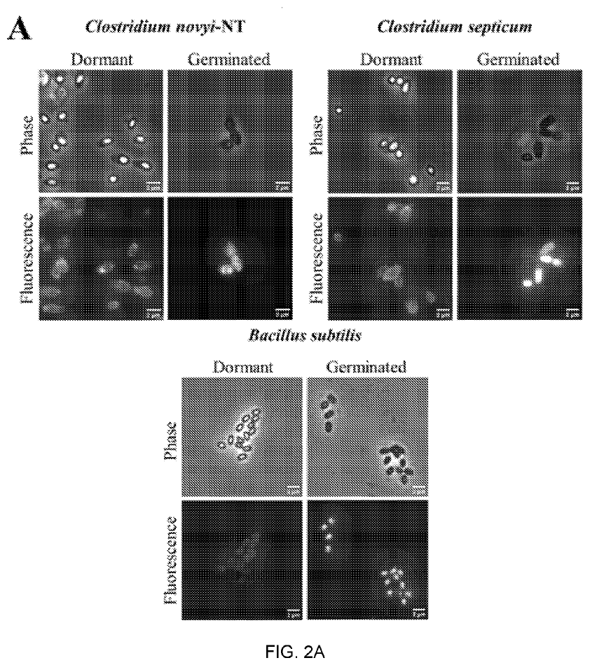

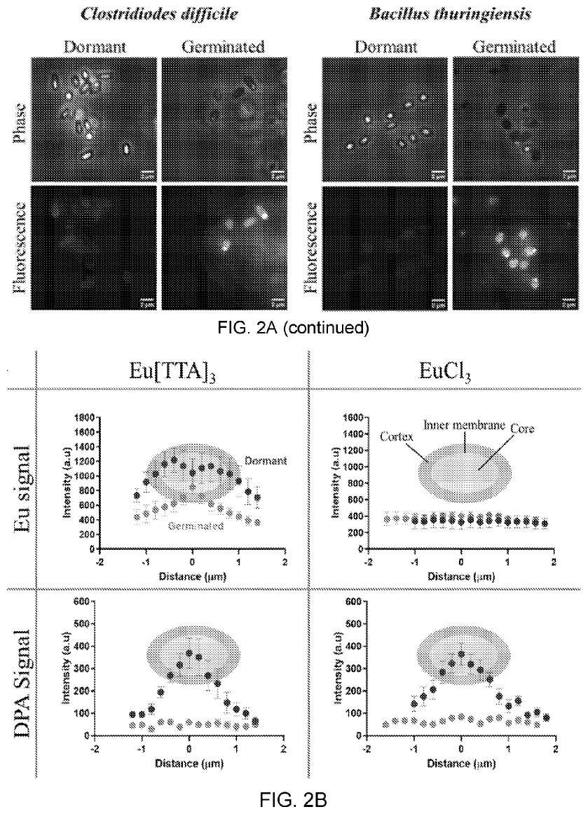

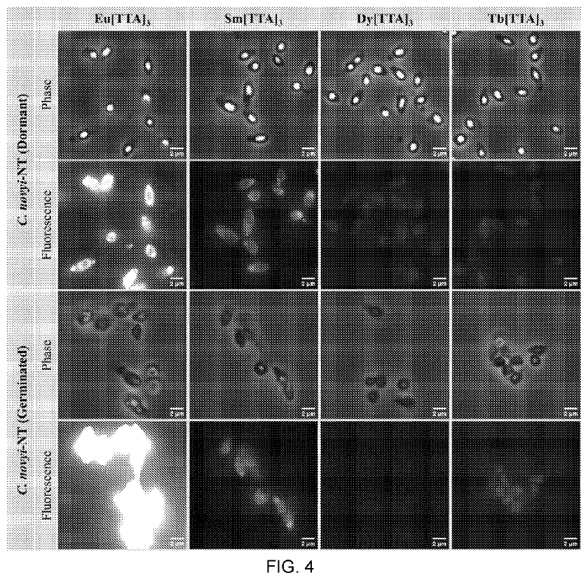

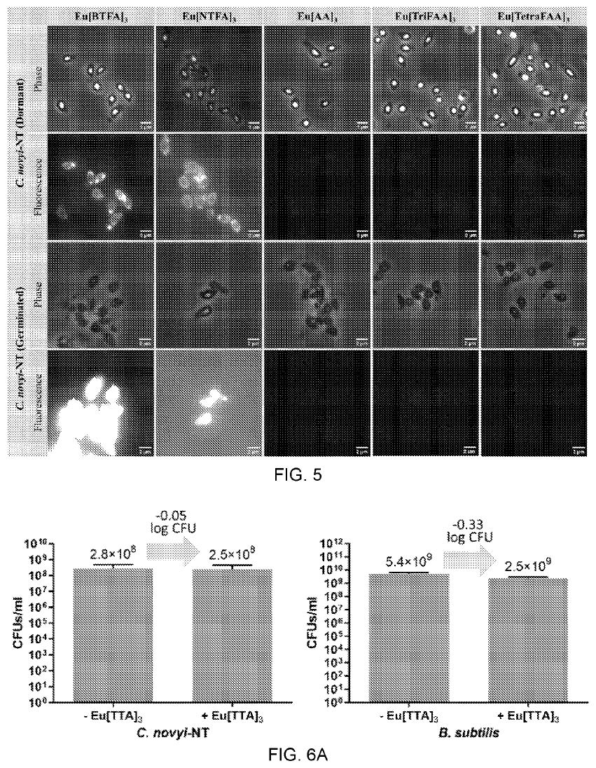

FIG. 2A shows that when the Ln-BD complex is Eu[TTA]₃, it stained both dormant and germinated spores of all bacteria tested with dormant spores sporting donut-shaped fluorescence harboring a central hollow, which became filled with bright luminescence post-germination. FIG. 4 shows that when the Ln-BD complex is Eu[TTA]₃, Sm[TTA]₃, Dy[TTA]₃, or Tb[TTA]₃ it similarly stained dormant C. novyi-NT spores and turned their cores fluorescent after germination. Further, germination resulted in brighter fluorescence within the core among all spores tested. Additionally, FIG. 5 illustrates that when the Ln-BD complex is Eu[BTFA]₃, Eu[NTFA]₃, Eu[AA]₃, Eu[TriFAA]₃, or Eu[TetraFAA]₃, aromatic BDs used in the Ln-BD complex stained dormant and germinated C. novyi-NT spores brighter when compared to Ln-BD complexes using aliphatic BDs. These results indicate that various Ln[BD]ₙ complexes can be used to stain a variety of bacterial spores. The results also demonstrate that aliphatic BDs are also useful for obtaining spore fluorescence.

The germinant dye solution for live cell imaging of C. novyi-NT spores was prepared by mixing appropriate amounts of the germinants L-cysteine (1M, 53.5 μl) and hypoxanthine (0.001 M, 53.5 μl), Oxyrase (10 μl) for inducing hypoxia conditions for germination and Eu[TTA]₃ (0.0015 M, 383 μl). To an aliquot (50 μl) of this germination solution, C. novyi-NT spore solution (5×10⁹ CFU/ml, 3.5 μl) is added such that the final concentrations of L-cysteine, hypoxanthine and Eu[TTA]₃ were 100 mM, 0.1 mM and 1.1 mM respectively. 10 μl of this solution was added on to a Superfrost Plus microscope glass slide, covered with glass coverslip (#1.5) and sealed on all four sides using nail polish. For B. subtilis spores, the germinant dye solution was prepared by mixing appropriate amounts of L-Alanine (100 mM, 107 μl) and Eu[TTA]₃ (0.0015 M, 393 μl). The heat-treated spores were allowed to adhere onto a normal slide at 4° C. for 1 hr and the germinant dye solution (50 μl) was then added to the adhered spores and immediately sealed with a coverslip and imaged. The final concentrations of L-alanine and Eu[TTA]₃ were 20 mM and 1.1 mM respectively.

For the live cell imaging alone, a cage incubator attached to the microscope was set to 37° C. and the germination was tracked at this temperature. The images were captured using the Metamorph Series 7.7(M 7.7 ) software. Imaging parameters are as follows: Binning 1, Exposure time—100 ms (phase contrast mode and fluorescence images) unless otherwise indicated. All images for the different types of bacterial spores were acquired under the same incident light intensity settings. All images were cropped and processed using ImageJ Version 1.53n. All the time-lapse images were first corrected for XY drift over time using the Stackreg Plugin in ImageJ prior to cropping.

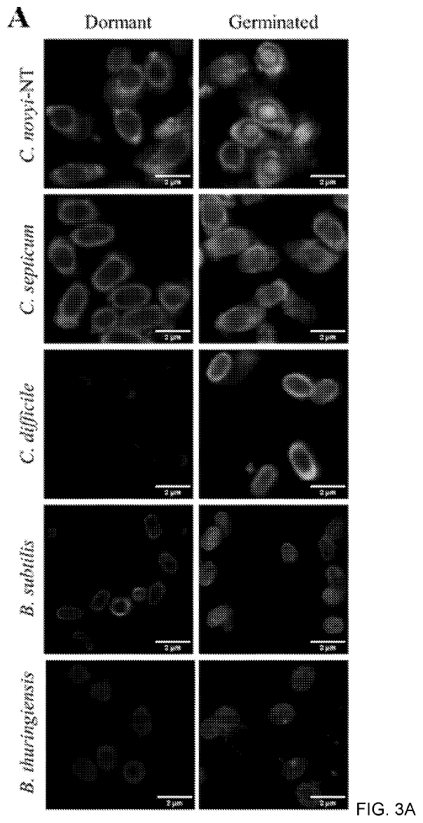

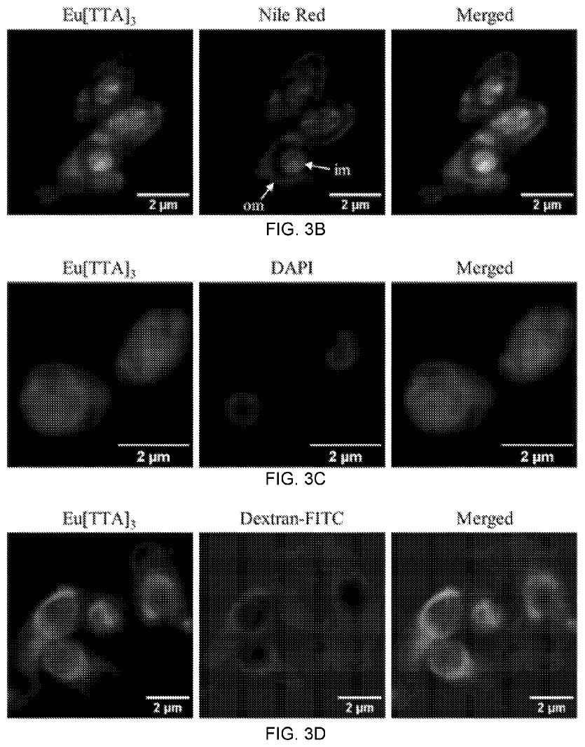

As shown in FIG. 3A, confocal images with Eu[TTA]₃ alone, the slides were imaged using a Leica SP8 super resolution microscope with a 100× HCX PL APO Objective, (N.A. 1.4, Oil objective lens), 405 nm Diode laser, Detection at 580-700 nm, 0.5AU pinhole. The images were acquired through the Leica Application Suite X 1.8 (LASX v 1.8) software and deconvoluted using the lightning mode with same parameters for all bacteria except for Bacillus spores where an optimization factor of 3 was used instead of 1.

All confocal images were cropped and processed using ImageJ Version 1.53n. The videos were generated using the 3D maximum projection function of Z-stack images obtained using the confocal microscope and processed using ImageJ.

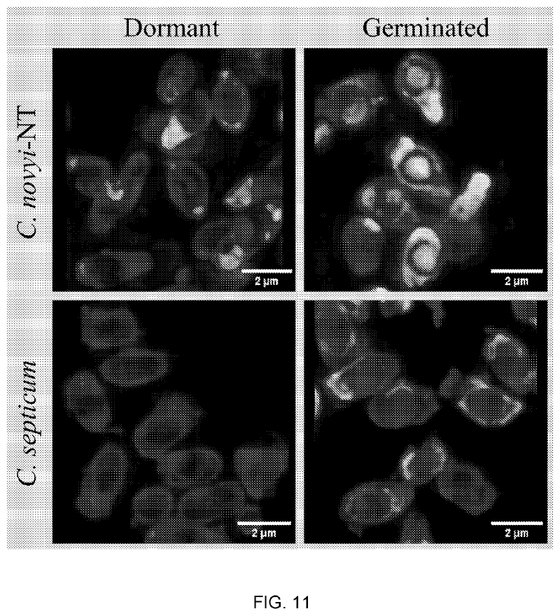

As shown in FIGS. 3A and 11, optical plane slice images through the dormant spores stained with Eu[TTA]₃ of all bacteria tested showed a hollow devoid of fluorescent signal which became fluorescent upon germination.

### Example 4

Raman imaging of germination of Clostridium novyi-NT spores. In order to better elucidate the effects of the present invention, Raman microscopy was used as a tool to map distribution of the Eu. The germinant dye solution for Raman live cell imaging of C. novyi-NT spores was prepared by mixing appropriate amounts of the germinants L-Cysteine (1M, 160.5 μl) and Hypoxanthine (0.001 M, 53.5 μl), Oxyrase (10 μl) and Eu[TTA]₃ (0.0015 M, 276 μl). A higher concentration of germinants compared to the other experiments was used to facilitate rapid germination to counter the probable photo damage of the spores due to the laser over time. To an aliquot (50 μl) of this germination solution, C. novyi-NT spore solution (5×10⁹ CFU/ml, 3.5 μl) was added so that the final concentrations of L-cysteine, hypoxanthine and Eu[TTA]₃ or EuCl₃ were 300 mM, 0.1 mM, and 0.77 mM, respectively. 10 μl of this solution is added on to a Quartz microscope glass slide (Photonik, Singapore) and covered with Quartz coverslip (#1.5) and sealed on all four sides using nail polish. The population germination studies were carried out by placing the slide over a heating stage set to 37° C. (Linkam Controller DC95) while the X-mapping studies were carried out at room temperature without a heating stage.

Simultaneous phase contrast, fluorescence and Raman imaging was accomplished using a Nikon Eclipse Ci microscope fitted with a PhotoFluor LM 75 fluorescence unit, 532 nm laser and a uRaman spectrometer (Einst Technologies, Singapore). The phase contrast and fluorescence images were captured using a CoolSnap HQ2 monochrome camera. The objective used was a 100× Nikon Plan Ph3 DL objective (N.A. 1.25, Oil objective lens) and a DAPI filter (Excitation filter—350/5 BP; Emission filter—412 LP) was used for the fluorescence imaging.

Images were captured using the Metamorph software and Raman spectra were obtained using the uSoft software (Einst Technologies, Singapore). Raman spectra were processed using the uRaman process software (Einst Technologies, Singapore) and the baseline was corrected for all the samples using the same parameters by the Savitzky-Golay method. The fluorescence images alone were processed further for deconvolution via CMLE method using the Hyugens Professional v16.10 software.

FIG. 2B shows the distribution of Eu along the longest axis of C. novyi-NT spores treated either with Eu[TTA]₃ or uncomplexed Eu (in the form of EuCl₃). C. novyi-NT spores were used for this study because of its larger size relative to the other bacteria used in this study allowing for high resolution mapping to be performed. Raman microscopy not only provides evidence independent of fluorescence about the localization of Eu in spores, but also allows for the simultaneous detection of the spore biomarker Dipicolinic Acid (DPA). As shown in the bottom row of FIG. 2B, DPA (1017 cm⁻¹) was hence observed to be concentrated within the core during dormancy and germination resulted in spore DPA concentrations which were no higher than the environment, showing release from the core. This DPA distribution profile was true regardless of whether spores were treated with Eu[TTA]₃ or EuCl₃.

In contrast to the DPA profiles, Eu[TTA]₃ and EuCl₃ produced vastly different results in dormant and germinated spores, as illustrated by the top row of FIG. 2B. Spores treated with Eu[TTA]₃ showed a double peak profile, with the peaks coinciding with the inner membrane region and a trough within the core region. Upon germination, the Eu signal spatially redistributed to form a single peak within the core, corroborating the bright core fluorescence observed post-germination. Additionally, there is no net gain in intra-spore Eu after germination, yet spore core fluorescence appears brighter to the eye relative to dormant spores.

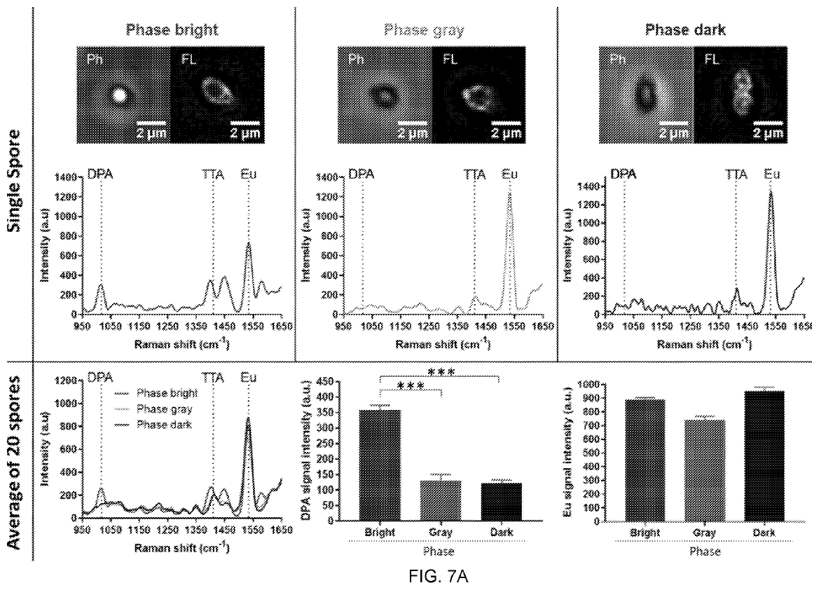

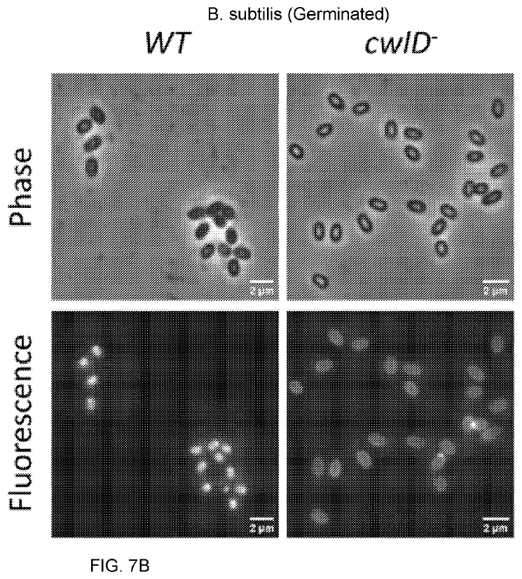

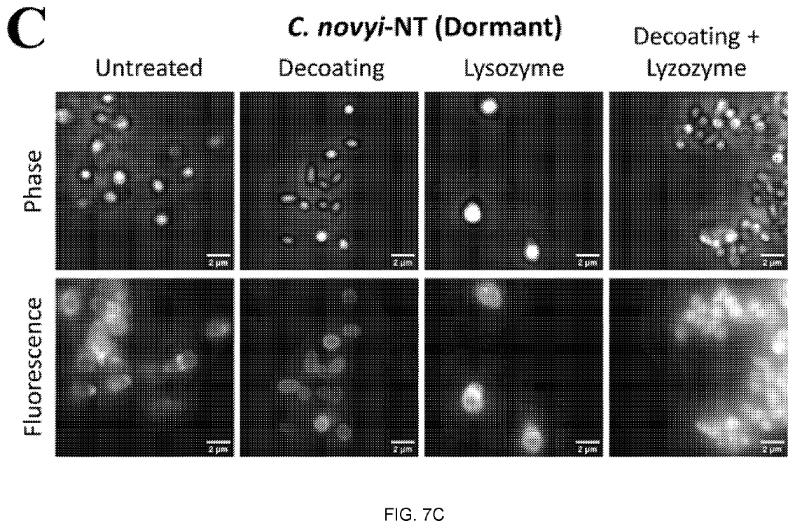

To interrogate the relationship of DPA and Eu[TTA]₃ fluorescence distribution in bacterial spores, C. novyi-NT spores from all three phase stages (bright, gray, dark) were imaged, with 20 spores analyzed per phase stage. Each spore was first stage-classified by phase contrast, then imaged under fluorescence and subsequently analyzed by Raman spectroscopy as described above. The Raman laser was aimed at the center of the spore to generate the general Raman profile of the spore. Data for a typical spore is shown in FIG. 7A. Phase bright spores exhibited a characteristic DPA peak at Raman shift 1017 cm⁻¹ as well as donut fluorescence. In phase gray spores, this DPA peak was now absent, implying DPA release, whereas the donut pattern was still observed. Phase dark spores lacked the DPA peak as expected, but now exhibited spore core fluorescence. These results demonstrate that DPA release precedes Eu[TTA]₃ fluorescence in the core. Additionally FIG. 7A shows that, there is no statistically significant shift in the Raman profile of Eu levels between phases, suggesting that the quantity of Eu in a spore remains generally stable throughout germination.

### Example 5

Co-staining assays. Germinated C. novyi-NT spores were incubated with Eu[TTA]₃ (1.4 mM) and Nile red (0.2 μg/ml) or DAPI (2.5 μM, 30 min) or FITC-Dextran 3 k-5 k (0.45 mg/ml). Aliquots from these samples were used for imaging. Slides were imaged using a Leica SP8 super resolution microscope with a 100× HCX PL APO Objective, (N.A. 1.4, Oil objective lens). Images were acquired using the Leica Application Suite X 1.8 (LASX v 1.8) software and deconvoluted using the lightning mode.

FIGS. 3B-3D illustrate that Ln[BD]ₙ complexes can be used in combination with other dyes in order to examine additional elements of spore morphology. Co-staining with Nile Red labeled the inner and outer spore membranes which are the lipid-rich regions in the spore, as shown in FIG. 3B. The inner membrane staining by Nile Red overlapped strongly with Eu[TTA]₃ fluorescence although Eu[TTA]₃ fluorescence extended beyond the inner membrane boundary to fill the spore core delineated by the Nile Red-stained inner membrane. Co-staining with the DNA-intercalating agent DAPI revealed a crystalline nucleoid of DNA shaped like a comma, as shown in FIG. 3C. This comma structure nested squarely within the spore core as defined by Eu[TTA]₃ fluorescence. Co-staining with FITC-Dextran (molecular weight 3-5 kD) revealed that the spore outer membrane was still intact as a physical barrier and hence impermeable to FITC-Dextran, as shown in FIG. 3D. In contrast, Eu[TTA]₃, with its smaller molecular size and lipophilic character, is able to penetrate the outer membrane and to stain the inner membrane as well as the core.

### Example 6

Viability assays. C. novyi-NT spores were germinated as described previously in Example 3 and Bacillus subtilis was germinated at 93.5 mM L-alanine. To 10 ul aliquots of the germinated spores, Eu[TTA]₃ (1.5 mM, 142.8 ul) was added and incubated at room temperature for 15 min so that the final concentration was 1.4 mM. The spores were then plated onto BHI (10%)FBS agar plates inside the Plas Labs anaerobic chamber for C. novyi-NT or LB agar plates plated aerobically for B. subtilis. Colonies were counted the following day.

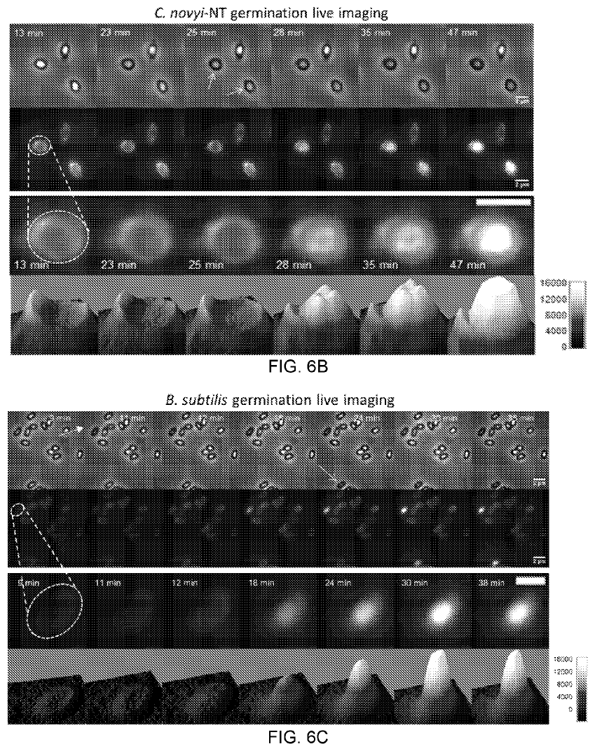

FIG. 6A shows that treatment with Eu[TTA]₃ did not result in any statistically significant loss in viability, with both C. novyi-NT and Bacillus subtilis retaining their outgrowth and colony-forming abilities.

Since Eu[TTA]₃ did not affect viability of spores, spore germination was followed in real time. Spores were mounted on a slide and incubated at 37° C. using appropriate germinants for C. novyi-NT (FIG. 6B) and B. subtilis spores (FIG. 6C). Time-lapse phase contrast and fluorescence microscopy were then performed according to the conditions outlined in Example 3 to capture incipient spore germination. Spores started out phase bright and with donut-shaped fluorescence. This fluorescence pattern remained unaltered as the spore became phase gray. It was only upon entry into the phase dark stage that the donut hollow turned fluorescent. This process happened within the span of 10-20 minutes, rendering the core brighter in fluorescence than the donut fluorescence which circumscribed it.

In the case of C. novyi-NT, incipient fluorescence in the core was observed as a pattern of punctate fluorescent points which grew in size to eventually cover the entire spore core, as shown in FIG. 6B. B. subtilis followed the same train of events, with a hollow spore core which was then rapidly filled with fluorescence upon entry into phase darkening, as shown in FIG. 6C.

### Example 7

Cortex degradation study. To study the role of the cortex layer of bacterial spores in potentially promoting or impeding the penetration of Lanthanide-beta-diketones into the spore core, the following study was performed. For C. novyi-NT cortex removal, dormant spores were treated with de-coating buffer (0.09M NaOH/0.9 % SDS/0.09M DTT/0.09 M NaCl) for 1 hour at 37° C.. After 6 times washing the spore in sterile water to remove the de-coating buffer, the spores were then treated with cortex lysis buffer (1× Halt™/50 mM Tris pH 7.4/2 mg/ml lysozyme/ 40 μg/ml MgCl₂) at 37° C. for 30 min. The spores were again washed with water and re-suspended in Tris-Halt™-MgCl₂ buffer (which was cortex lysis buffer without the lysozyme component) or 1×PBS for further analysis. An aliquot of spores at each stage was set aside for imaging. cwlD mutant B. subtilis spores which lack muramic acid-8-lactam essential for cortex hydrolysis, were germinated and stained with Eu[TTA]₃ according to the methods described in Example 3.

Microscopy was done on an inverted wide-field microscope. For C. novyi-NT spores, imaging conditions were: Leica DMI 6000 with a Hamamatsu ORCA flash 4.0 LT camera. The imaging was done at 1000× magnification with a long pass DAPI filter. The exposure time was 50 ms for both modes except for the fluorescence images of dormant and de-coated spores which was at 100 ms. For B. subtilis spores, the conditions were: Zeiss Axiovert 200M, photometrics Coolsnap HQ2 camera and 1000× magnification long pass DAPI filter and 100 ms exposure time for both modes.

As shown in FIG. 7B, Eu[TTA]₃ failed to stain the core of cwlD mutant B. subtilis germinated spores with intact cortexes, showing that the cortex restricted access to Eu[TTA]₃. In contrast, FIG. 7C shows that bright core fluorescence was observed when dormant C. novyi -NT spores were treated with both the de-coating and lysozyme steps, as described above, but not if treated with either alone. These results show that full removal of all layers before the inner membrane allowed Eu[TTA]₃ access to the dormant spore core and that cortex hydrolysis is necessary for Eu[TTA]₃ to access the spore core.

### Example 8 (Comparative Examples)

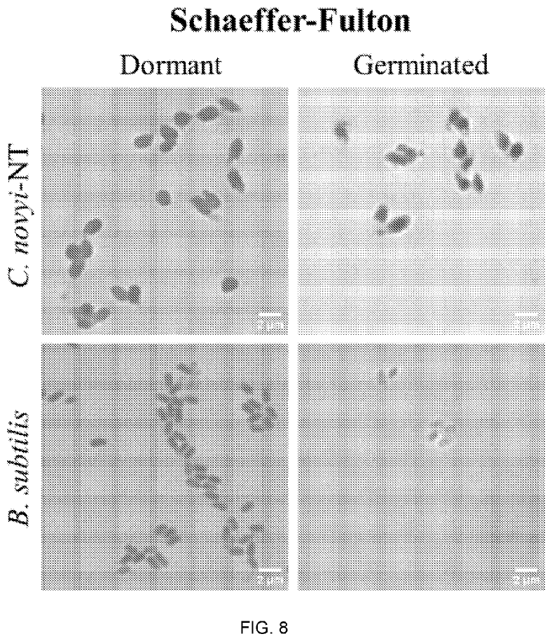

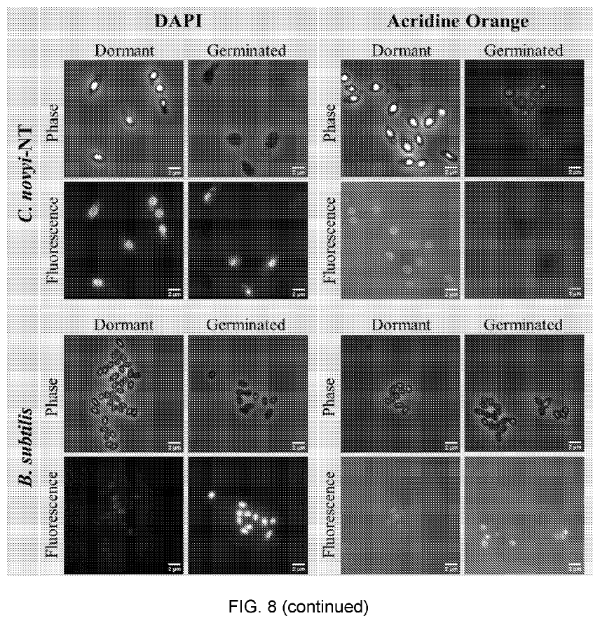

DAPI staining. Both C. novyi-NT and B. subtilis spores (dormant and germinated) were incubated with DAPI (5 μM) and aliquots from these samples were used for imaging, as shown in FIG. 8 (Bottom, left column). The imaging was done using a wide-field inverted Zeiss Axiovert 200M microscope with a 100× Plan Apochromat Ph3 objective (N.A. 1.4, Oil objective lens) and a longpass DAPI filter fitted with a Photometrics Coolsnap HQ2 monochrome camera. The images were captured using the Metamorph Series 7.7(M 7.7 ) software. All image processing and cropping was done using ImageJ.

Acridine orange staining. For C. novyi-NT spores, dormant spores or germinated spores (3.5 μl) were incubated with acridine orange solution (50 ul, 10 mg/ml) for 30 min (final concentration 9.3 mg/ml) and then imaged, as shown in FIG. 8 (Bottom, right column, row 1). For Bacillus subtilis 1 A1 wild-type spores, the heat treated dormant and germinated spore slides were prepared as described in the previous section following which acridine orange solution was added and the slides were immediately imaged, as shown in FIG. 8 (Bottom, right column, row 2). The imaging was done using a widefield inverted Zeiss Axiovert 200M microscope with a 100× Plan Apochromat Ph3 objective (N.A. 1.4, Oil objective lens) and an EGFP filter (Excitation filter-470/40 BP; Dichroic 495; Emission filter 525/50 BP) fitted with a Coolsnap HQ2 monochrome camera. The images were captured using the Metamorph Series 7.7 (M 7.7 ) software. All image processing and cropping was done using ImageJ.

Malachite green staining. Both C. novyi-NT and B. subtilis spores (dormant and germinated) were heat fixed on a heating block at 50° C. for 5 min, following which Malachite green solution (5%) was constantly added over the heat-fixed smear covered with a filter paper and placed in a water bath steaming at 95° C. for 15 min. The slides were then rinsed with deionized water to remove the excess malachite green stain and then counter stained with Safranin for 5 min. The slides were finally again rinsed with water and blotted with paper towels. They were then imaged using a Zeiss Axioplan 2 upright microscope fitted with a 100× Plan Apochromat Ph3 objective lens (1.4 N.A., Oil objective lens) and a Leica DFC 7000T color camera, as shown in FIG. 8 (Top). The images were captured using the Leica Application Suite X 1.8 (LASX v 1.8) software. Imaging parameters: Exposure time—50 ms. The images were processed using the minimum filter with a 2 pixel radius. All image processing and cropping was done using ImageJ.

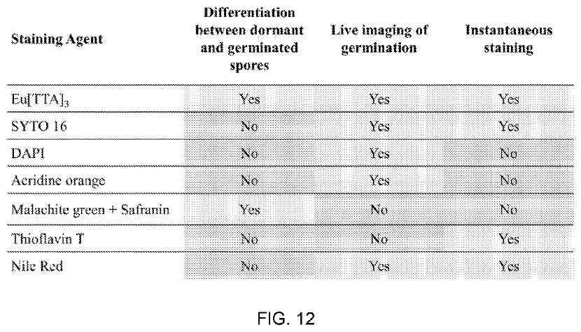

The Schaeffer-Fulton method uses malachite green and a safranin counterstain to distinguish between dormant and germinated forms. Sample processing is long and destroys spore viability, hence precluding live imaging. DAPI stained both dormant and germinated spores with high fluorescence but did not differentiate between them. Finally, AO provided some distinction between dormant and germinated spores, but failed to stain germinated spores visibly. Both DAPI and AO required an incubation time of at least 30 minutes to achieve sufficient contrast in staining. A comparison of the performance of various staining agents can be found in FIG. 12.

### Example 9

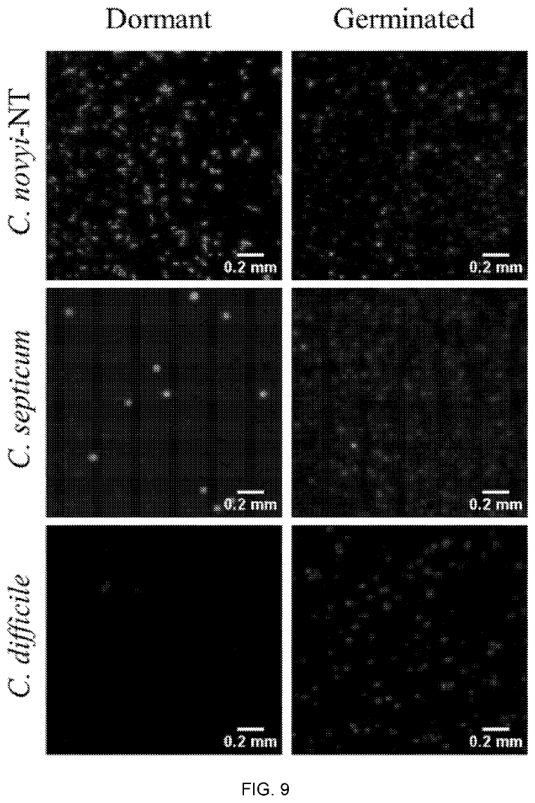

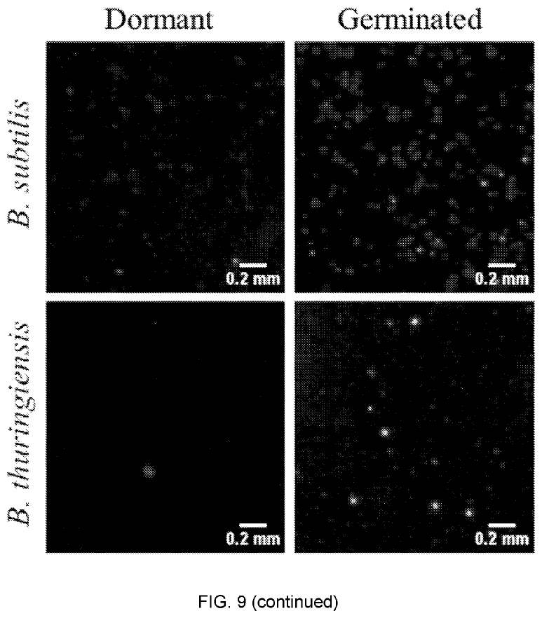

Dino-lite imaging. Eu[TTA]₃ stained bacterial samples were prepared for dormant and end-point germinated spores as in Example 3. The images were obtained using a Dino-Lite Premier digital microscope AM 4113T with DAPI excitation at 55× magnification. Images were cropped and processed using ImageJ. FIG. 9 shows that, despite the low resolution of the images, spores stained with Eu[TTA]₃ were observed to be significantly more fluorescent compared to the background.

### Example 10

Milk matrix experiments. A 5% w/v non-fat milk solution was prepared by dissolving blotting grade milk powder in water. Equal volumes of dormant C. novyi-NT spores were added to this solution such that the final concentration of milk was 2.5% (w/v) and spores was 2.5×10⁹ CFU/ml. An aliquot (3.5 l) of this sample was then stained with Eu[TTA]₃ (1.4 mM). Samples were imaged using a Zeiss inverted Axio Observer 7 wide-field microscope, 1600× magnification with long pass DAPI filter. The exposure time was 100 ms for the phase contrast mode and 50 ms for the fluorescence mode. The display histogram was adjusted differently for each acquisition mode to ensure maximum visibility.

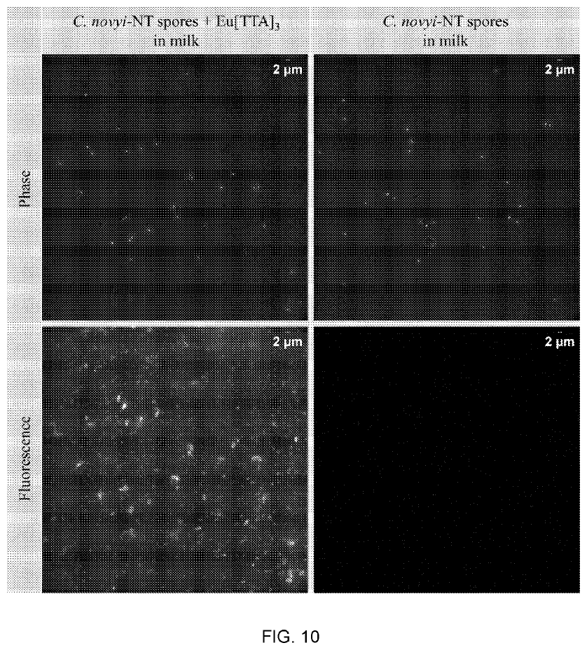

FIG. 10 illustrates that spores were clearly visualized under fluorescence only in the presence of Eu[TTA]₃ and it was easy to make out the shape donut-shaped fluorescence of the milk-suspended spores even in the presence of background fluorescence.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations are not intended to convey that the claimed subject matter requires features or combinations of features other than those expressly recited in the claims. Accordingly, the scope of this disclosure is intended to include all modifications and variations encompassed within the spirit and scope of the following appended claims.

### Example 11 (complex Mixture Samples)

Spore outgrowth staining: C. novyi-NT dormant spores were incubated anaerobically (37° C., 7 hours) in media containing L-Cysteine (100 mM), Hypoxanthine (0.1 mM), BHI (0.62×), FBS (9.3% v/v), Oxyrase (1:50 v/v). B. subtilis dormant spores were first heat activated in 1×PBS (70° C., 1 hour) followed by incubation in LB media aerobically (37° C., 4 hours). The media were then removed by centrifugation (3900 rcf, 15 min) followed by addition of Eu[TTA]₃ (1.4 mM). Aliquots of these samples were then imaged.

Sporulating culture staining: Vegetative B. cereus cells were cultured aerobically (37° C., overnight to allow early sporulation) in 2×SG sporulation medium with shaking at 220 rpm (Infors HT Multitron Standard shaking incubator) and harvested by centrifugation (3900 rcf, 15 min) the next day after 24 hours. Cells were then stained with Eu[TTA]₃ (1.4 mM) and imaged.

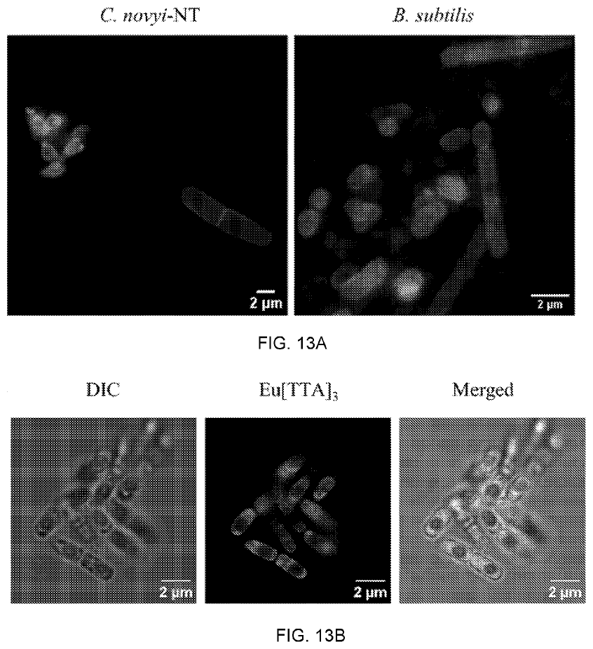

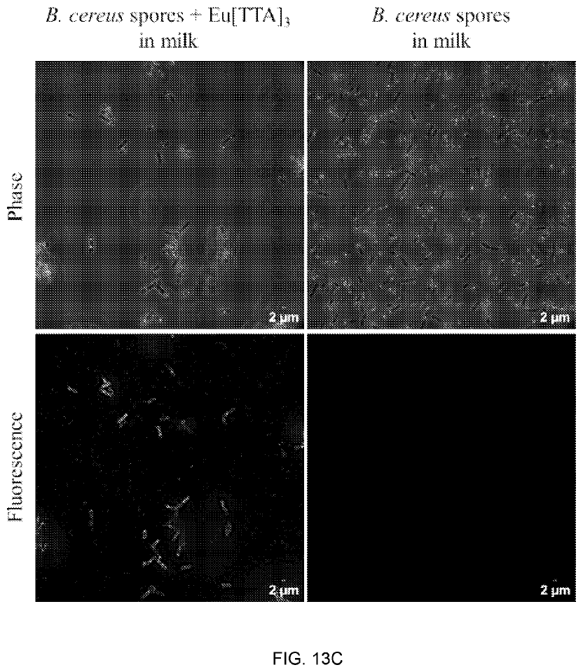

In natural environments, co-mingling of spores with vegetative bacteria is to be expected. With this in consideration, staining was performed on samples derived either from spore outgrowth (FIG. 13A) or overnight cultures of sporulating vegetative bacteria (FIG. 13B). In cultures of C. novyi-NT or B. subtilis, spores exhibited brighter fluorescence compared to their vegetative counterparts and were easily distinguished based on their morphology and size. A similar trend was observed in early sporulating cultures of B. cereus, where the developing forespore of sporulating cells was marked by a hollow region, highlighting the utility of Eu[TTA]₃ in spore visualization irrespective of the presence of vegetative forms.

Milk matrix experiments: A 5% w/v non-fat milk solution was prepared by dissolving blotting grade milk powder in water. Equal volumes of dormant B. cereus spores were added to this solution such that the final concentration of milk was 2.5% (w/v) and spores was 1.75×10¹⁰ CFU/ml (B. cereus). An aliquot (3.5 μl) of this sample was then stained with Eu[TTA]₃ (1.4 mM) and imaged.

Finally, the ability of Eu[TTA]₃ to address spore detection in food matrices was also studied. The issue of dairy contamination by sporulating bacteria such as B. cereus and C. botulinum is well-documented, for example in Christiansson, A. ; Bertilsson, J. ; Svensson, B.

Bacillus Cereus Spores in Raw Milk: Factors Affecting the Contamination of Milk during the Grazing Period. J. Dairy Sci. 1999, 82 (2), 305-314; Gupta, T. B. ; Brightwell, G. Farm Level Survey of Spore-Forming Bacteria on Four Dairy Farms in the Waikato Region of New Zealand. Microbiologyopen 2017, 6 (4); Barash, J. R.; Hsia, J, K.; Arnon, S. S. Presence of Soil-Dwelling Clostridia in Commercial Powdered Infant Formulas. J. Pediatr. 2010, 156 (3), 402-408. Such contamination was simulated by inoculating milk with spores of B. cereus followed by imaging with and without Eu[TTA]₃ (FIG. 13C). Despite moderate background fluorescence, visible fluorescent spores sporting hollow cores were easily visualized only when Eu[TTA]₃ was present. These results demonstrate the potential of Eu[TTA]₃ for spore detection in complex food matrices.

### Numbered Aspects

Notwithstanding the appended claims, the following numbered aspects also form part of the instant disclosure and are also examples and representative species of the present invention.
1. A method for live imaging one or more bacterial spores in a sample, the method comprising:
contacting the sample with a probe having a formula Ln[BD]ₙ wherein Ln is a member of the lanthanide series of elements, BD is a beta-diketone, and n is a whole number integer, to stain one or more bacterial spores in the sample; and
detecting a fluorescent signal from the one or more stained bacterial spores in the sample using a fluorescence microscope.
2. A method for distinguishing germination states of one or more bacterial spores in situ, the method comprising:
contacting a sample comprising one or more bacterial spores with a probe having a formula Ln[BD]ₙ, wherein Ln is a member of the lanthanide series of elements, BD is a beta-diketone, and n is a whole number integer, to stain one or more bacterial spores in the sample;
performing signal detection by live imaging fluorescent signal of the sample using a fluorescence microscope; and
distinguishing between dormant bacterial spores and germinating or germinated bacterial spores in the sample;
wherein dormant bacterial spores have a fluorescence signal characterized by a donut-shaped fluorescence profile with a central hollow, and
wherein germinating or germinated bacterial spores have a central core filled with fluorescence.
3. A method for real-time live imaging germination of bacterial spores, the method comprising:
contacting one or more bacterial spores with a germinant probe solution comprising one or more germinants and a probe having a formula Ln[BD]ₙ, wherein Ln is a member of the lanthanide series of elements, BD is a beta-diketone, and n is a whole number integer, to stain one or more bacterial spores in the sample to form a mixture;
mounting the mixture on a slide;
visualizing the morphology of the one or more bacterial spores on the slide using time-lapse phase-contrast microscopy over time; and
imaging fluorescent signals from the one or more bacterial spores on the slide using time-lapse fluoresce microscopy over time,
wherein the real-time live imaging of the germination of the one or more bacterial spores comprises three phase-contrast microscopy phases comprising: 1) a phase bright stage and a 2) phase gray stage of spore germination correlated with the most intense fluorescent signal being localized to regions exterior to the core of the spore, and 3) a phase dark stage of spore germination correlated with the most intense fluorescent signal being localized to the core region of the spore, wherein the phase dark stage indicates bacterial spore germination.
4. The method of any one or combination of numbered aspects disclosed herein, wherein the signal detection is performed using confocal microscopy,
wherein the optical plane slice images through dormant spores show a hollow region devoid of any fluorescent signal corresponding to the core region of the spore, and
wherein the optical plane slice images through germinated spores show a fluorescent signal in the center of the spore corresponding to the core region of the spore.
5. The method of any one or combination of numbered aspects disclosed herein, wherein the germinants are one or more of L-cysteine, hypoxanthine, oxyrase, sodium glycocholate, taurocholate, glycine, L-alanine, L-valine, D-valine, or D-cycloserine.
6. The method of any one or combination of numbered aspects disclosed herein, wherein the Ln in the Ln[BD]ₙ probe is Europium, Samarium, Dysprosium, or Terbium.
7. The method of any one or combination of numbered aspects disclosed herein, wherein the BD in the Ln[BD]ₙ probe is at least one selected from the group consisting of thenoyltrifluoroacetone (TTA), 1-(-2-Naphthoyl)-3,3,3-trifluoroacetone (NTFA), 4,4,4-Trifluoro-1-phenyl-1,3-butanedione (BTFA), Acetylacetone (AA), 1,1,1 Trifluoroacetylacetone (TriFAA), and 1,1,5,5 Tetrafluoroacetylacetone (TetraFAA).
8. The method of any one or combination of numbered aspects disclosed herein, wherein n in the Ln[BD]ₙ probe is 1 to 5.
9. The method of any one or combination of numbered aspects disclosed herein, wherein n in the Ln[BD]ₙ probe is 2 to 4.
10. The method of any one or combination of numbered aspects disclosed herein, wherein n in the Ln[BD]ₙ probe is 3.
11. The method of any one or combination of numbered aspects disclosed herein, wherein the probe is at least one selected from the group consisting of Eu[TTA]₃, Sm[TTA]₃, Dy[TTA]₃, Tb[TTA]₃, Eu[BTFA]₃, Eu[NTFA]₃, Eu[AA]₃, Eu[TriFAA]₃, and Eu[TetraFAA]_{3.}
12. The method of any one or combination of numbered aspects disclosed herein, wherein the probe binds to spores from at least one of the Bacillus genera, the Clostridium genera, or the Clostridioides genera.
13. The method of any one or combination of numbered aspects disclosed herein, where the probe binds spores from at least one of B. subtilis, B. thuringiensis, B. cereus, C. novyi-NT type A, C. difficile, or C. septicum.
14. The method of any one or combination of numbered aspects disclosed herein, comprising performing signal detection using a USB powered microscope, a digital microscope, a confocal microscope, a Raman microscope, or a bright-field microscope.
15. The method of any one or combination of numbered aspects disclosed herein, wherein treatment with the probe does not significantly affect spore viability, outgrowth, or colony-forming ability when compared to untreated spores.
16. The method of any one or combination of numbered aspects disclosed herein, wherein a change in colony forming units (CFU) of the bacterial spores before and after treatment with the probe is less than 0.5 log CFU.
17. The method of any one or combination of numbered aspects disclosed herein, wherein staining with the probe occurs instantaneously or near instantaneously.
18. The method of any one or combination of numbered aspects disclosed herein, wherein the method further comprises contacting the sample with one or more additional probes or dyes and detecting one or more additional signals.
19. The method of any one or combination of numbered aspects disclosed herein, wherein the ratio of Ln to BD in the Ln[BD]ₙ probe is in the range of 9:1 to 1:9.
20. The method of any one or combination of numbered aspects disclosed herein, wherein the concentration of the probe is in the range of 0.7 mM to 10 mM.
21. The method of any one or combination of numbered aspects disclosed herein, wherein the concentration of the probe is in the range of 1 mM to 5 mM.
22. The method of any one or combination of numbered aspects disclosed herein, wherein the concentration of the probe is in the range of 1.4 mM to 2.8 mM.
23. The method of any one or combination of numbered aspects disclosed herein, wherein the concentration of the probe is about 1.4 mM or about 2.8 mM.
24. The method of any one or combination of numbered aspects disclosed herein, wherein the method does not include a step of spore permeabilization or a step of heating.
25. The method of any one or combination of numbered aspects disclosed herein, wherein spore cortex hydrolysis precedes probe entry into the core.
26. The method of any one or combination of numbered aspects disclosed herein for use in food pathogen detection, environmental monitoring, quality assurance, or microbiology research. In some aspects, the food is milk.
27. The method of any one or combination of numbered aspects disclosed herein, comprising using Raman microscopy to distinguish dormant spores showing a double peak profile from germinated spores showing a single peak profile.
28. The method of any one or combination of numbered aspects disclosed herein, comprising detecting peaks in the double peak profile comprising an inner membrane region and a trough between the peaks coinciding with core region of the spore, and comprising detecting the single peak profile comprising the core of the spore.
29. The method of any one or combination of numbered aspects disclosed herein, wherein there is no statistically significant shift in the Raman profile of the Ln levels of the Ln[BD]ₙ probe throughout germination.
30. The method of any one or combination of numbered aspects disclosed herein, comprising detecting Dipicolinic Acid (DPA) is not present when the single peak corresponding to the Ln[BD]ₙ probe in the core region of the spore is present.
31. The method of any one or combination of numbered aspects disclosed herein, wherein the probe is Europium (III) thenoyltrifluoroacetone (Eu[TTA]₃).
32. The method of any one or combination of numbered aspects disclosed herein, wherein the fluorescence microscope is equipped with a 4′,6-diamidino-2-phenylindole (DAPI) long-pass filter.
33. The method of any one or combination of numbered aspects disclosed herein, further comprising adding one or more surfactants to the sample.
34. The method of any one or combination of numbered aspects disclosed herein, wherein the one or more surfactants are 2-[4-(2,4,4-trimethylpentan-2-yl)phenoxy]ethanol, a polysorbate, sodium dodecyl sulfate, or a combination thereof.
35. The method of any one or combination of numbered aspects disclosed herein, further comprising co-staining bacterial spores with Nile Red, DAPI, fluorescein isothiocyanate (FITC)-dextran, or a combination thereof.
36. The method of any one or combination of numbered aspects disclosed herein, comprising monitoring spore bioburden in food or pharmaceutical samples, monitoring spore bioburden in the environment, or monitoring spore bioburden as a quality assurance step in an industrial process.
37. The method of any one or combination of numbered aspects disclosed herein, wherein the industrial process is in producing self-healing concrete.
38. The method of any one or combination of numbered aspects disclosed herein, wherein the sample comprises a plurality of bacterial spores, and the method further comprises quantifying the plurality of bacterial spores in the sample.
39. The method of any one or combination of numbered aspects disclosed herein, comprising visualizing stained bacterial spores immediately after the contacting step.
40. The method of any one or combination of numbered aspects disclosed herein, wherein the imaging and/or monitoring comprises producing a video of the sample to observe changes in germination over time.
41. The method of any one or combination of numbered aspects disclosed herein, wherein a fluorescent brightener is not used.
42. The method of any one or combination of numbered aspects disclosed herein, wherein the method does not involve a permeabilization step.
43. A kit comprising a germination probe having a formula Ln[BD]ₙ wherein Ln is a member of the lanthanide series of elements, BD is a beta-diketone, and n is a whole number integer in a first container, and a germinant in a second container.
44. The kit of any one or combination of numbered aspects disclosed herein, wherein the germinant is L-cysteine, hypoxanthine, oxyrase, sodium glycocholate, taurocholate, glycine, L-alanine, L-valine, D-valine, or D-cycloserine.
45. The kit of any one or combination of numbered aspects disclosed herein, wherein the kit comprises a plurality of germinants, wherein each germinant is provided in a separate container.
46. A composition comprising a germination probe having a formula Ln[BD]ₙ wherein Ln is a member of the lanthanide series of elements, BD is a beta-diketone, and n is a whole number integer, and a germinant.
47. The composition of any one or combination of numbered aspects disclosed herein, wherein the germinant is L-cysteine, hypoxanthine, oxyrase, sodium glycocholate, taurocholate, glycine, L-alanine, L-valine, D-valine, or D-cycloserine.
48. A live-imaging apparatus for use in live imaging one or more bacterial spores in a sample in which the one or more bacterial spores have been contacted with a probe having a formula Ln[BD]ₙ wherein Ln is a member of the lanthanide series of elements, BD is a beta-diketone, and n is a whole number integer, to stain one or more bacterial spores in the sample, comprising a live imaging capture device configured to detect staining of the one or more bacterial spores in the sample.
49. An apparatus for distinguishing germination states of one or more bacterial spores in a sample in situ, wherein the sample comprises one or more bacterial spores stained with a probe having a formula Ln[BD]ₙ, wherein Ln is a member of the lanthanide series of elements, BD is a beta-diketone, and n is a whole number integer, wherein the apparatus is configured to obtain a live image fluorescent signal of the sample using fluorescence microscopy and distinguish between dormant bacterial spores and germinating or germinated bacterial spores in the sample, wherein dormant bacterial spores have a fluorescence signal characterized by a donut-shaped fluorescence profile with a central hollow, and wherein germinating or germinated bacterial spores have a central core filled with fluorescence.
50. An apparatus for real-time live imaging germination of bacterial spores that have been contacted with a germinant probe solution comprising one or more germinants and a probe having a formula Ln[BD]ₙ, wherein Ln is a member of the lanthanide series of elements, BD is a beta-diketone, and n is a whole number integer, to stain one or more bacterial spores in the sample to form a mixture, wherein the apparatus comprises a slide configured to hold said mixture, wherein the apparatus comprises a time-lapse phase-contrast microscope configured to visualize the bacterial spores in the mixture and image fluorescent signals from the bacterial spores on the slide, wherein the real-time live imaging of the germination of the one or more bacterial spores comprises three phase-contrast microscopy phases comprising: 1) a phase bright stage and a 2) phase gray stage of spore germination correlated with the most intense fluorescent signal being localized to regions exterior to the core of the spore, and 3) a phase dark stage of spore germination correlated with the most intense fluorescent signal being localized to the core region of the spore, wherein the phase dark stage indicates bacterial spore germination.
51. Use of the method of any one or combination of numbered aspects disclosed herein for monitoring spore bioburden in food or pharmaceutical samples, for monitoring spore bioburden in the environment, or for monitoring spore bioburden as a quality assurance step in an industrial process.
52. Use of the method of any one or combination of numbered aspects disclosed herein for a quality assurance step in producing self-healing concrete.
53. Use of the method of any one or combination of numbered aspects disclosed herein for quantifying a plurality of bacterial spores in the sample.
54. Use of the method of any one or combination of numbered aspects disclosed herein for visualizing stained bacterial spores immediately after the contacting step.
55. Use of the method of any one or combination of numbered aspects disclosed herein for producing a video of the sample to observe changes in germination over time.
56. Use of the kit of any one or combination of numbered aspects disclosed herein in a method according to any one of any one or combination of numbered aspects disclosed herein.
57. Use of the composition of any one or combination of numbered aspects disclosed herein in a method according to any one of any one or combination of numbered aspects disclosed herein.

While the subject matter of this disclosure has been described and shown in considerable detail with reference to certain illustrative aspects, including various combinations and sub-combinations of features, those skilled in the art will readily appreciate other aspects and variations and modifications thereof as encompassed within the scope of the present disclosure. Moreover, the descriptions of such aspects, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims. Section headings, the materials, methods, and examples are illustrative only and not intended to be limiting.

Other aspects, advantages, and modifications are within the scope of the following claims.

1. A method for live imaging one or more bacterial spores in a sample, the method comprising:
contacting the sample with a probe having a formula Ln[BD]ₙ wherein Ln is a member of the lanthanide series of elements, BD is a beta-diketone, and n is a whole number integer, to stain one or more bacterial spores in the sample; and
detecting a fluorescent signal from the one or more stained bacterial spores in the sample using a fluorescence microscope.

2. A method for distinguishing germination states of one or more bacterial spores in situ, the method comprising:
contacting a sample comprising one or more bacterial spores with a probe having a formula Ln[BD]ₙ, wherein Ln is a member of the lanthanide series of elements, BD is a beta-diketone, and n is a whole number integer, to stain one or more bacterial spores in the sample;
performing signal detection by live imaging fluorescent signal of the sample using a fluorescence microscope; and
distinguishing between dormant bacterial spores and germinating or germinated bacterial spores in the sample;
wherein dormant bacterial spores have a fluorescence signal characterized by a donut-shaped fluorescence profile with a central hollow, and
wherein germinating or germinated bacterial spores have a central core filled with fluorescence.

3. A method for real-time live imaging germination of bacterial spores, the method comprising:
contacting one or more bacterial spores with a germinant probe solution comprising one or more germinants and a probe having a formula Ln[BD]ₙ, wherein Ln is a member of the lanthanide series of elements, BD is a beta-diketone, and n is a whole number integer, to stain one or more bacterial spores in the sample to form a mixture;
mounting the mixture on a slide;
visualizing the morphology of the one or more bacterial spores on the slide using time-lapse phase-contrast microscopy over time; and
imaging fluorescent signals from the one or more bacterial spores on the slide using time-lapse fluoresce microscopy over time,
wherein the real-time live imaging of the germination of the one or more bacterial spores comprises three phase-contrast microscopy phases comprising: 1) a phase bright stage and a 2) phase gray stage of spore germination correlated with the most intense fluorescent signal being localized to regions exterior to the core of the spore, and 3) a phase dark stage of spore germination correlated with the most intense fluorescent signal being localized to the core region of the spore, wherein the phase dark stage indicates bacterial spore germination.

4. The method of claim 2, wherein the signal detection is performed using confocal microscopy,
wherein the optical plane slice images through dormant spores show a hollow region devoid of any fluorescent signal corresponding to the core region of the spore, and
wherein the optical plane slice images through germinated spores show a fluorescent signal in the center of the spore corresponding to the core region of the spore.

5. The method of claim 3, wherein the germinants are one or more of L-cysteine, hypoxanthine, oxyrase, sodium glycocholate, taurocholate, glycine, L-alanine, L-valine, D-valine, or D-cycloserine.

6. The method of claim 1, wherein the Ln in the Ln[BD]ₙ probe is Europium, Samarium, Dysprosium, or Terbium.

7. The method of 3claim 1, wherein the BD in the Ln[BD]ₙ probe is at least one selected from the group consisting of thenoyltrifluoroacetone (TTA), 1-(-2-Naphthoyl)-3,3,3-trifluoroacetone (NTFA), 4,4,4-Trifluoro-1-phenyl-1,3-butanedione (BTFA), Acetylacetone (AA), 1,1,1 Trifluoroacetylacetone (TriFAA), and 1,1,5,5 Tetrafluoroacetylacetone (TetraFAA).

8. The method of claim 1, wherein n in the Ln[BD]ₙ probe is 1 to 5.

9. (canceled)

10. (canceled)

11. The method of claim 1, wherein the probe is at least one selected from the group consisting of Eu[TTA]₃, Sm[TTA]₃, Dy[TTA]₃, Tb[TTA]₃, Eu[BTFA]₃, Eu[NTFA]₃, Eu[AA]₃, Eu[TriFAA]₃, and Eu[TetraFAA]₃.

12. The method of claim 1, wherein the probe binds to spores from at least one of the Bacillus genera, the Clostridium genera, or the Clostridioides genera.

13. The method of claim 12, where the probe binds spores from at least one of B. subtilis, B. thuringiensis, B. cereus, C. novyi-NT type A, C. difficile, or C. septicum.

14. The method of claim 1, comprising performing signal detection using a USB powered microscope, a digital microscope, a confocal microscope, a Raman microscope, or a bright-field microscope.

15. The method of claim 1, wherein treatment with the probe does not significantly affect spore viability, outgrowth, or colony-forming ability when compared to untreated spores, or wherein a change in colony forming units (CFU) of the bacterial spores before and after treatment with the probe is less than 0.5 log CFU, or wherein staining with the probe occurs instantaneously or near instantaneously.

16. (canceled)

17. (canceled)

18. The method of claim 1, wherein the method further comprises contacting the sample with one or more additional probes or dyes and detecting one or more additional signals.

19. The method of 3claim 1, wherein the ratio of Ln to BD in the Ln[BD]ₙ probe is in the range of 9:1 to 1:9, or wherein the concentration of the probe is in the range of 0.7 mM to 10 mM.

20. (canceled)

21. (canceled)

22. (canceled)

23. (canceled)

24. The method of claim 1, wherein the method does not include a step of spore permeabilization or a step of heating.

25. The method of claim 1, wherein spore cortex hydrolysis precedes probe entry into the core.

26. (canceled)

27. The method of claim 2, comprising using Raman microscopy to distinguish dormant spores showing a double peak profile from germinated spores showing a single peak profile.

28. The method of claim 27, comprising detecting peaks in the double peak profile comprising an inner membrane region and a trough between the peaks coinciding with core region of the spore, and comprising detecting the single peak profile comprising the core of the spore, or wherein there is no statistically significant shift in the Raman profile of the Ln levels of the Ln[BD]ₙ probe throughout germination, or comprising detecting Dipicolinic Acid (DPA) is not present when the single peak corresponding to the Ln[BD]ₙ probe in the core region of the spore is present.

29. (canceled)

30. (canceled)

31. The method of claim 1, wherein the probe is Europium (III) thenoyltrifluoroacetone (Eu[TTA]₃), or wherein the fluorescence microscope is equipped with a 4′,6-diamidino-2-phenylindole (DAPI) long-pass filter.

32. (canceled)

33. The method of claim 1, further comprising adding one or more surfactants to the sample.

34. The method of claim 33, wherein the one or more surfactants are 2-[4-(2,4,4-trimethylpentan-2-yl)phenoxy]ethanol, a polysorbate, sodium dodecyl sulfate, or a combination thereof.

35. The method of claim 1, further comprising co-staining bacterial spores with Nile Red, DAPI, fluorescein isothiocyanate (FITC)-dextran, or a combination thereof, or monitoring spore bioburden in food or pharmaceutical samples, monitoring spore bioburden in the environment, or monitoring spore bioburden as a quality assurance step in an industrial process.

36-57. (canceled)
